(19) United States
(12) Patent Application Publication
(10) Pub. No.: US 2014/0171490 A1
(43) Pub. Date: Jun. 19, 2014

(54) Method and Device for Ophthalmic Administration of Active Pharmaceutical Ingredients

(71) Applicant: PHARMALIGHT INC., Wilmington, DE (US)

(72) Inventors: Yossi Gross, Moshav Mazor (IL); Rafi Herzog, Bat-Shlomo (IL); Steven B. Koevary, Newton, MA (US)

(73) Assignee: PHARMALINGT INC., Wilmington, DE (US)

(21) Appl. No.: 13/963,991

(22) Filed: Aug. 9, 2013

Related U.S. Application Data
(62) Division of application No. 11/883,774, filed on Aug. 6, 2007, now abandoned.
(60) Provisional application No. 60/650,144, filed on Feb. 7, 2005.
(60) Provisional application No. 60/742,870, filed on Dec. 7, 2005.

(30) Foreign Application Priority Data
Feb. 6, 2006 (IL) PCT/IL2006/000145

(51) Int. Cl. A61K 47/46; A61K 31/498; A61K 31/496; A61K 31/222; A61K 31/573; A61K 47/48; A61K 31/575; A61K 31/138; A61K 31/4704; A61K 31/5377; A61K 31/433; A61K 31/542; A61K 31/382; A61K 31/5575; A61K 9/00; A61K 31/4168

(52) U.S. Cl. CPC A61K 47/46; A61K 31/4168; A61K 31/498; A61K 31/496; A61K 31/222; A61K 31/573; A61K 47/48215; A61K 31/575; A61K 31/138; A61K 31/4704; A61K 31/5377; A61K 31/433; A61K 31/542; A61K 31/382; A61K 31/5575; A61K 9/0048
USPC 514/44 R; 514/392; 514/249; 514/253.04; 514/548; 514/179; 514/182; 514/652; 514/312; 514/546; 514/236.2; 514/363; 514/226.5; 514/432; 514/622; 514/530; 514/777

(57) ABSTRACT

Disclosed is the use of a mist of a pharmaceutical composition for ophthalmic delivery of a protein or peptide active pharmaceutical ingredient, a related method of treatment and a device useful in implementing the use and method. Disclosed is also the use of a mist for ophthalmic delivery of a pharmaceutical composition including a highly irritating penetration enhancer and an ophthalmically acceptable carrier, a related method of treatment and a device useful in implementing the use and method. Disclosed is also a device for ophthalmic administration configured to direct a mist of a pharmaceutical composition to the eye only when the eye is open. Disclosed is also a self-sterlizing device for ophthalmic administration. Disclosed is also a device and a method for increasing the bioavailability of an ophthalmically administered API in a pharmaceutical composition.

### FIELD AND BACKGROUND OF THE INVENTION

The present invention relates to the field of medicine and more particularly, to methods and devices relating to ophthalmic administration of pharmaceutical compositions including an active pharmaceutical ingredient (API) to a patient.

The bulb of the eye (bulbus oculi; eyeball) is contained in the cavity of the orbit, where it is protected from injury. Associated with the eye are certain accessory structure such as the muscles, fasciae, eyelids, conjunctiva, and lacrimal apparatus. Only the surface of the anterior part of the eye, including the corneal epithelium and part of the episcleral conjunctiva, are exposed to the environment. The mucosa of the conjunctiva provide a protective interface between the eye and accessory structures. The exposed anterior surface is continuously washed by tear fluid. The nasolacrimal duct drains tears and other substances from the eye to be absorbed by a layer of mucosal membrane.

In the art, ophthalmic administration of a pharmaceutical composition including an active pharmaceutical ingedient is known. Most commonly, ophthalmic administration of a pharmaceutical composition is for ocular delivery via a corneal or scleral route. That said, systemic delivery of an API by ophthalmic administration of a pharmaceutical composition via the conjunctival route (including the mucosa of the eyelids and nasolacrimal duct) is also known.

Ophthalmic administration of a pharmaceutical composition is challenging for a number of reasons, see for discussion Burrows J. et al. Drug Deliv. Comp. Rep. 2002, spring. As discussed below, the eye is a sensitive organ with an easily damaged surface. There is rapid elimination of an applied composition due to lacrimation and drainage through the nasolacrimal duct. APIs are neutralized by binding to, or metabolization by, tear proteins.

There are many modes of ophthalmic administration of pharmaceutical compositions. The most common mode of ophthalmic administration is by instillation of drops using an eye-dropper or other device, see for example U.S. Pat. Nos. 5,152,435; 6,336,917; 6,386,394; 6,401,979; 6,447,476; 6,547,770; 6,610,036 and RE38,077.

Although technically simple, instillation of eye drops has many disadvantages. Receiving eye drops requires practice; it is unpleasant to open an eye widely while the drop is instilled, for adults but especially for childre. Self-administration is not simple and often not effective when a drop is innaccurately placed. Often a person will instill more than the required number of drops, whether by accident or intent, and drops have a notoriously poorly defined volume making accurate dosage virtually impossible (Lederer, C. M. Jr. et al. Am. J. Opthalmol. 1986, 101(6), 691-694 reports between 25 and 56 ul). Inadvertent contact of an eye dropper with the eye occurs, potentially damaging the eye and compromising sterility.

As noted above, much of an ophthalmically administered pharmaceutical composition is washed out or drained away, and much of the API is neutralized by the ocular protective mechanisms. Eye drops, by applying a seemingly wastefully large amount of pharmaceutical composition, overcome the challenges posed by ophthalmic administration. Although much of an administered composition is washed away, drains away and even leaks out along the face, enough remains for a long enough time to be effective. The massive volume of pharmaceutical composition washes away the tear fluids and dilutes the concentration of the tear proteins. Further, the seemingly excessive amount of API ensures that even if some API is bound to tear proteins or metabolized by the proteins, enough API remains potent to exercise a desired pharmaceutical effect. Thus, although seemingly wasteful and difficult to accurately dose, eye drops in fact provide a simple and effective route for ophthalmic administration.

An additional mode of ophthalmic administration is by the use of a nebulizer that transforms a pharmaceutical composition into a mist that is then contacted with exposed portions of the eye. Devices described as producing mists effective for ophthalmic administration of pharmaceutical compositions include those described in U.S. Pat. Nos. 4,052,985; 5,203,506; 5,893,515; 6,062,212, 6,530,370 and “Nanotechnology News from the University of Minnesota”, Fall 2005, p. 7 Ophthalmic administration using a mist has the advantage of accurate dosing and economical use. That said, the required device for such administration is relatively complex (compared to an eye dropper). Further, as the volumes of pharmaceutical composition actually delivered are relatively small, the tear fluid effectively washes away such compositions as delivered. Further, as the rate of API delivery is relatively small (in terms of molecules per unit time), the eye has sufficient time to bind to and metabolize administered susceptible APIs. Devices for nebulizing pharmaceutical compositions for ophthalmic administration to the eye are well known to one skilled in the art.

Peptide and protein APIs are well-known in the field of medicine. One of the challenges of using peptide and protein APIs is administration. Like with any API, systemic administration by injection (whether intramuscular, subcutaneous or into the circulatory system) of a pharmaceutical composition including a peptide or protein API is unpleasant, especially for treatment of chronic medical condidtions that require regular and repeated administration, for example the treatment of diabetes mellitus with insulin. Further, many peptide and proteins are potentially effective as APIs if delivered to specific sites within the body, for example specific organs such as the brain or central nervous system, but systemic administration by injection is inefficient or ineffective. A peptide or protein API injected into the body is susceptible to degradation by proteolytic enzymes found in the circulation system. In order to ensure that a sufficient amount of peptide or protein arrives at a target organ or specific location in the body, a large amount of peptide or protein must be administered. Further, peptides and proteins cannot penetrate the blood brain barrier, precluding the use of peptides and proteins systemically administered via injection for treatment of the brain and central nervous system.

In the art, systemic administration of peptides and protein APIs via the conjunctiva using eye drops is known, see Koevary, S. B. Curr. Drug. Metab 2003, 4(3) 213-222; Morgan, R. V. J. Ocular Pharm. Ther. 1995, 11(4), 565-573 (Insulin Saettone, M. F. et al. Int. J. Pharma. 1996, 142, 103-113 (beta-blocking agents); Ke, T. L. et al. Inflammation 2000, 24(4), 371-384; Sasaki et al. J. Pharm Pharmacol 1994, 46(11), 871-875 (insulin); U.S. Pat. No. 5,182,258 (peptides and small proteins up to 6 kDa) and references therein.

There are many advantages to systemic administration via the conjunctiva of peptides and protein APIs relative to systemic administration via injection, including ease of use, patient comfort, safety and simpler self-administation. However, as the conjunctival route is systemic, administered peptide and protein APIs are exposed to enzymatic degradation and there exist locations in the body, such as the nervous system and brain, which are not accessible to a systemically administered peptide or protein API.

As discussed above, instillation of eye drops is a wasteful mode of ophthalmic administration, flooding the eye with an excessive volume of pharmaceutical composition and an excessive amount of API, but it is the wastefulness that provides eye drops with particular efficacy. Thus, eye drops have a disadvantage for use in the delivery of peptide and protein APIs that are quite expensive. However, alternative modes of ophthalmic administration of a pharmaceutical composition including a peptide or protein API are less suitable. For example, the use of a nebulizer to administer a pharmaceutical composition including a peptide or protein API as a mist is expected to be ineffective.

The delivery of peptides and proteins, especially larger peptide and protein APIs by mist cannot be expected to succeed. As is known, the activity of larger peptides and proteins is determined by a specific three-dimensional structure. Modification of the structure causes the peptide or protein to lose activity or even change in activity. Whereas the secondary structure of a peptide or protein is largely determined by the amino acid sequence, tertiary structure is largely determined by the environment in which the peptide or protein is found, especially salts and solvents. During nebulization, a significant amount of energy is tranferred into a pharmaceutical composition. The energy is expected to heat each individual mist particle to the extent that a peptide or protein held therein is denaturated. Further, the heat and the large surface are of the nebulized pharmaceutical composition causes evaporation of solvent molecules from the mist particles, increasing the concentration of salts and additives in the mist particles. This high concentration is expected to be of the extent that a peptide or protein held therein is denatured.

Further, as a class, peptide and protein APIs are more susceptible to metabolization and binding than small molecule APIs, so when applied more gradually and in lesser amounts, as with a mist mode of delivery, the peptide or protein will be more quickly neutralized. As a result, the mist mode is expected to be ineffective both for systemic delivery and for ocular delivery of a peptide or protein API.

There is a lack of an effective and economical alternative to drops as a method of administration of peptide and protein APIs, for delivery in a pharmaceutically effective form to a desired site within the body, especially to the central nervous system.

Topical administration of APIs, to surfaces such as the skin, mucous membranes, conjunctiva, sclera and cornea is well-known in the field of medicine. Generally, a pharmaceutical composition is formulated in such a way that when applied to a surface, the included API penetrates into or through the surface. To increase penetration of a topically applied APIs, penetration enhancers are often added to topical pharmaceutical compositions. Penetration enhancers act by various mechanisms to increase the permeability of a surface to an API.

An exceptional challenge in the field of medicine is the use of penetration enhancers in ophthalmic pharmaceutical compositions, whether to increase the permeability of the conjunctiva, sclera or cornea. Generally, effective penetration enhancers are irritants that cause severe ocular damage. In Morgan, R. V. J. Ocular Pharm. Ther. 1995, 11(4), 565-573 is reported that saponin and Brij-99 are strongly irritation to the eye. In Saettone, M. F. et al. Int. J. Pharma. 1996, 142, 103-113 is reported that saponin, escin, digitonin, BL-9, benzalkonium chloride and sodium deoxycholate are strongly irritating to the eye. In Furrer, P. et al. AAPS PharmSci 2002, 4(1), 1-5 is reported that saponin and sodium fusidate are strongly irritating to the eye.

As a rsulet, less effective penetration enhancers are used. However, the amount of such less effective penetration enhancers in an ophthalmic pharmaceutical composition must be kep relatively low to prevent ocular damage and are conseuqnely of limied efficacy. In Morgan, R. V. J. Ocular Pharm. Ther. 1995, 11(4), 565-573 is reported the use of 0.5% Brij-78 (polyoxyethylene(20) steryl ether) as a penetration enhancer in an ophthalmic pharmaceutical composition. In Saettone, M. F. et al. Int. J Pharma. 1996, 142, 103-113 is reported the use of 1% sodium ursodeoxycholate, 2% Brij 78, 1% sodium taurodeoxycholate, 2% sodium tauroursodeoxycholate, 0.5% Brij 35 and 0.5% EDTA as penetration enhancers in ophthalmic pharmaceutical compositions. In Furrer, P. et al. AAPS PharmSci 2002, 4(1), 1-5 is reported the use of 1% DMSO, 1% decamethonium bromide, 1% Tween 20, 1%Brij 35, 1% EDTA, 1% sodium glycocholate and 1% sodium cholate as penetration enhancers in ophthalmic pharmaceutical compositions. In Burgalassi, S. et al. Tox. Lett. 2001, 122, 1-8 is reported that benzalkonium chloride and cetylpyridinium chloride are less toxic to human corneal epithelial cells than Brij-78 but more toxic than EDTA, while polyethoxylated castor oil is less toxic than EDTA.

There is a general lack of a method to allow the use of more effective penetration enhancers, (i.e., the use of more effective but irritating penetration enhancers such as saponin or of higher amounts of less irritating penetration enhancers such as EDTA) for increasing the efficacy of ophthalmic pharmaceutical compositions including an API.

In the field of medicine it is known that the treatment of chronic conditions often necessitates administration of an API repeatedly, often on a multiple daily basis. As API administration by a health-care professional is generally expensive as a result of the cost of the health-care professional and the cost of transporting the health-care professional to the patient, self-administration of an API is preferrd for a person who needs repeated administration of an API to treat a chronic condition. The most convenient method of self-administration of an API is using an orally administrable API, for example using a pill or capsule, but many APIs are not orally available. Other methods of administration may require an expensive administration device, may provide inaccurate dosing and may be unpleasant or inconvenient. For example, administration devices duch as insulin pumps or spring-loaded syringes are expensive and complex. Eye drops, nose drops and other transmucosal administration methods provide highly variable dosages both due to the variability in the amount of API-containing pharmaceutical composition and to variability in amounts of API entering the body. Administration by injection, eye drops or inhalation is often unpleasant, reducing patient quality of life and compliance.

In the fild of medicine it is recognized that there is often a need for the administration of an API to a large group of people, for example for administration of vaccines or other prophylactic APIs or for administration of APIs for the treatment of epidemics, pandemics or endemic conditions. In such high-throughput administration situations, it is necessary that the health-care professional actually administering the API spends as little time as possible per patient. At the same time, issues of sterility and accurate dosage cannot be comprised. Known methods are insufficient. Many APIs are not suitable for intramuscular administration using a transdermal spray-device. As noted above, many APIs cannot be orally administered and it is difficult to ensure that a varied population, for example including the young, elderly or uneducated actually takes the orally administered API. Injections require disposable administration devices to ensure absolute sterility, require highly skilled health care professionals and are difficult to perform quickly due to ubiquitous needle phobia. Eye drops and inhalation devices are difficult to dose accurately and often cause discomfort to subjects, and sterility requires disposable devices.

There is a general lack of a high-throughput administration method that provides accurate dosing, is quick, causes little discomfort to a patient including young, elderly and frail and can be performed by a less-skilled health care professional.

It would be highly advantageous to have a method of administering peptide and protein APIs devoid of at least some of the disadvantages of the prior art.

It is desirable to increase the bioavailibility of ophthalmically administered APIs. A preferred method of increasing bioavailability of topically administered APIs, coadministration of a penetration enhancers with the API, is not useful due to the ocular irritation caused by effective penetration enhancers. There is a need for increasing the bioavailability of ophthalmically administered APIs. It would also be highly advantagous to have a method of ophthalmic administration of APIs employing penetration enhancers devoid of at least some of the disadvantages of the prior art.

It would also be highly advantageous to have a method of high-throughput administration of APIs devoid of at least some of the disadvantages of the prior art.

### SUMMARY OF THE INVENTION

The present invention successfully addresses at least some of the shortcomings of prior art by providing

According to the teachings of the present invention there is provided for the use of a mist of a pharmaceutical composition for ophthalmic delivery of an active pharmaceutical ingredient selected from the group consisting of proteins and peptides to a subjec in need thereof.

According to the teachings of the present invention there is also provided for the use of a mist of a pharmaceutical composition for ophthalmic delivery of an active pharmaceutical ingredient selected from the group consisting of proteins and peptides to a subject in need thereof.

According to the teachings of the present invention there is also provided for the use of a mist for ophthalmic delivery of a pharmaceutical composition including a highly irritating penetration enhancer and an ophthalmically acceptable carrier to a subject in need thereof.

In embodiments of the present invention the delivery is systemic.

In embodiments of the present invention the delivery is to the blood stream of the subject.

In embodiments of the present invention the delivery is to part of an eye of the subject, e.g., the sclera, the optic nerve and/or the retina.

In embodiments of the present invention the delivery is to part of the nervous system of the subject, e.g., the the brain, the central nervous system, the cerebral cavity, the cerebrospinal fluid, or the spinal cord.

According to the teachings of the present invention there is also provided a method of treatment, comprising: a) providing a pharmaceutical composition including an active pharmaceutical ingredient and an ophthalmically acceptable carrier; b) generating a mist of the composition; and c) contacting the mist with a posterior surface of an eye of a subject in need thereof thereby depositing an effective amount of the API on the posterior surface wherein the active ingredient is selected from the group consisting of peptides and proteins.

According to the teachings of the present invention there also is provided a method of delivering a composition, comprising: a) providing a pharmaceutical composition including a highly irritating penetration enhancer and an ophthalmically acceptable carrier; b) generating a mist of the composition; and c) contacting the mist with a posterior surface of an eye of a subject in need thereof.

In embodiments of the present invention a subject is a human.

In embodiments of the present invention a subject is a non-human animal.

In embodiments of the present invention the need is selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. Such conditions include, but are not limited to conditions such as behavioral conditions, brain disorders, cancer, eye cancers, brain cancers, cerebral cancers, nerve cancers, central nervous system disorders, choroidal neovascularization (e.g., associated with retinal or subretinal disorders, such as, age-related mascular degeneration, presumed ocular histoplasmosis syndrome, myopic degeneration, angioid streaks and ocular trauma), corneal neovascularization (e.g., associated with trauma, chemical burns or corneal transplantation), glaucoma, infections, inflammatory diseases, inflammations, inflammatory diseases of the retina, intravitreal neovascularization (e.g., associated with diabetic retinopathy, vein occlusion, sickle cell retinopathy, retinopathy of prematurity, retinal detachment, ocular ischemia and trauma), iris neovascularization (e.g., associated with diabetic retinopathy, vein occlusion, ocular tumor and retinal detachment), macular edema, mental illnesses, neural conditions, neurological disorders, ocular diseases, ocular inflammation, optic disc neovasvularization, optical nerve disorders, pannus posterior segment edema, postoperative ocular pain, proliferative vitreoretinopathy, prostaglandin formation, psychological conditions, psychoses and psychiatric disorders, pterygium, retinoblastoma, retinal edema, retinal degeneration, retinal revascularization (e.g., diabetic retinopathy, vein occlusion, sickle cell retinopathy, retinopathy of prematurity, retinal detachment, ocular ischemia and trauma), uveitis and vascular retinopathy.

In embodiments of the present invention, the condition is a condition susceptible to an interaction of an active pharmaceutical ingredient with a part of an eye, such as the cornea, retina, vitreos fluid, sclera, lens.

In embodiments of the present invention, the condition is a condition susceptible to an interaction of an active pharmaceutical ingredient with a nerve.

In embodiments of the present invention the condition is a condition susceptible to treatment with leptin or leptin homologues.

In embodiments of the present invention the condition is a condition susceptible to treatment with antibodies or antibody homologues, such as IgGl.

In embodiments of the present invention the condition is a condition susceptible to treatment with an aptamer, e.g., an anti-VEGF aptamer.

In embodiments of the present invention, the need requires delivery of an active ingredient to the blood stream of the subject.

In embodiments of the present invention, the need requires delivery of an active ingredient part of an eye of the subject, e.g., the cornea, retina, vitreous fluid, sclera, lens or optic nerve.

In embodiments of the present invention, the need requires delivery of an active ingredient to a part of the nervous system of the subject, e.g., the brain, the central nervous system, the cerebral cavity, the cerebrospinal fluid, an optic nerve, the retina and the spinal cord.

According to the teachings of the present invention, there is also provided a device for ophthalmic administration of a pharmaceutical composition, comprising: a) a nebulizer; b) a composition reservoir functionally associated with the nebulizer; and c) a pharmaceutical composition including an active pharmaceutical ingredient and an ophthalmically acceptable carrier contained with the reservoic wherein the active ingredient is selected from the group consisting of peptides and proteins.

According to the teachings of the present invention there is also provided a device for ophthalmic administration of a composition, comprising: a) a nebulizer; b) an composition reservoir functionally associated with the nebulizer; and c) a pharmaceutical composition including a highly irritating penetration enhancer and an ophthalmically acceptable carrier contained within the reservoir.

In embodiments of the present invention, a composition including a peptide or protein API further comprises a penetration enhancer. Suitable penetration enhancers include, but ar not limited to penetration enhancers selected from the group consisting of acetone, acyl lactylates, acyl peptides, acylsarcosinates, alcohols, alkanolamine salts of fatty acids, alkyl benzene sulphonates, alkyl ether sulphates, alkyl sulphates, allantoin, ammonium glycyrrhizide, anionic surface-active agents, 1-substituted azacycloheptan-2-ones, benzyl benzoate, bezyl salicylate, bile salts, Brij 35, Brij 78/35, butan-1,4-diol, butyl benzoate, butyl laurate, butyl myristate, butyl stearate, cationic surface-active agents, cetylpyridium chloride (mild) chenodeoxycholic acid, cholate, cholic acid, citric acid, cocoamidopropylbetaine, decamethonium, decamethonium bromide, decyl methyl sulfoxide, decyl oleate, deoxycholic acid, dibutyl azelate, dibutyl phthalate, dibenzyl sebacate, dibutyl sebacate, dibutyl suberate, dibuty succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, EDTA and disodium EDTA, ethyl caprate, ethyl caproate, ethyl caprylate, 2-ethyl-hexyl pelargonate, ethyl-2-hydroxypropanoate ethyl laurate, ethyl myristate, 1-ethyl-2-pyrrolidone, ethyl salicylate, fusidic acid, fusidate fusidic acid derivatives, glycerol monolaurate, hexyl laurate, glycocholate, glycocholic acid, glycodeoxycholic acid, glycyrrhizic acid, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, lecithin, maypons, metal salts of fatty acids, methy nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic thanolamide, pleyl alcohol, pentan-2,4-diol, phenoxyethanol, phosphatidyl choline, phosphine oxides, polyalkoxylated ether glycollates, poly(diallypiperidinium chloride), poly(dipropyldiallylammonium chloride), polyethylene glycol monolaurate, polyglycerol esters, polyoxyethylated castor oil (mild), polyoxyethylene, polyoxyethylene ethers of fatty acids such as polyoxyethylene 4-, 9-, 10-, and 23-lauryl ether, polyoxyethylene 10- and 20-cetyl ether, polyoxyethylene 10- and 20-stearyl ether, polyoxyethylene monolaurate, polyoxyethylene sorbitans such as polyoxyethylene sorbitan monolaurate, polyoxy:polyoxyethylene stearate, polyoxypropylene 15 stearyl ether, poly(vinyl pyridinium chloride), propan-1-ol, propan-2-ol, propylene glycol, propylene glycol dipelargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternized poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sodium cholate, sodium glycocholate, glycocholate, sodium deoxycholate, sodium taurocholate, sodium glycodeoxycholate, sodium taurodeoxycholate, sorbitan monooleate, sorbitan monolaurate, sugar esters, sulphosuccinate, taurocholic acid, taurodeoxycholic acid, tetrahydrofuran, tetrahydrofurfural alcohol, transcutol, triethanolamine dodecyl benzene sulphonate, triethanolamine oleate, TWEEN 20, urazole, urea, urosdeoxycholic acid, saponin, saponins and derivatives, esters, salts and mixtures thereof.

In embodiments of the present invention, a penetration enhancer is a penetration enhancer that is inherently highly irritating such as benzalkonium chloride, BL-9, deoxycholic acid. digitonin, escin, fusidic acid, fusidate, fusidic acid derivatives, saponin, saponins, sodium deoxycholate, acetone, acyl lactylates, acyl peptides, acylsarcosinates, alcohols, alkanolamine salts of fatty acids, alkyl benzene sulphonates, alkyl ether sulphates, alkyl sulphates, allantoin, anionic surface-active agents, 1-substituted azacycloheptan-2-ones, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, glycerol monolaurate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin lamepons, lauryl alcohol, lecithin, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyethylene glycol monolaurate, polyglycerol esters, poly(vinyl pyridinium chloride), propan-1-ol, propan-2-ol, propylene glycol, propylene glycol dipelargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternized poly (vinyl alcohol), sapamin hydrochloride, sodium cocaminopropionate, sodium dioctyl sulphonsuccinate, sodium laruate, sodium lauryl ether sulphate, sodium lauryl sulphate, sorbitan monooleate, sorbitan monolaurate, sugar esters, sulphosuccinate, tetrahydrofuran, tetrahydrofurfural alcohol, transcutol, triethanolamine dodecyl benzene sulphonate, triethanolamine oleate, urazole, urea, and derivatives, esters, salts and mixtures thereof. In embodiments of the present invention, such an inherently highly irritating penetration enhancer comprises at least 0.05%, at least 0.1%, at least 0.2%, at least 0.5%, at least 1%, at least 2% and even at least 3% by weight of the pharmaceutical composition.

In embodiments of the present invention the penetration enhancer is a penetration enhancer that is highly irritating at high concentrations such as ammonium glycyrrhizide, Brij 35, Brij 78, Brij-98, cetylpyridium chloride, chenodeoxycholic acid, cholate, cholic acid, decamethonium, decamethonium bromide, dimethyl sulphoxide, EDTA and disodium EDTA, glycocholate, glycocholic acid, glycodeoxycholic acid, glycyrrhizic acid, paraben, polyoxyethylene, polyoxyethylene ethers of fatty acids such as polyoxyethylene 4-, 9-, 10-, and 23-lauryl ether, polyoxyethylene 10- and 20-cetyl ether, polyoxyethylene 10- and 20-stearyl ether, polyoxyethylated castor oil, polyoxyethylene monolaurate, polyoxyethylene sorbitans such as polyoxyethylene sorbitan monolaurate, polyoxy:polyoxyethylene stearate, polyoxypropylene 15 stearyl ether, sodium cholate, sodium glycocholate, sodium taurocholate, sodium glycodeoxycholate, sodium taurodeoxycholate, sodium ursodeoxycholate, taurocholic acid, taurodeoxycholic acid, TWEEN 20, urosdeoxycholic acid, and derivatives, esters, salts and mixtures thereof in a greater than accepted concentration. In embodiments of the present invention, such a penetration enhancer that is highly irritating at high concentrations comprises at least 0.05%, at least 0.1%, at least 0.2%, at least 0.5%, at least 1%, at least 2%, at least 3% and even at least 4% by weight of the pharmaceutical composition.

In embodiments of the present invention, the penetration enhancer comprises saponin.

In embodiments of the present invention, a composition further comprises an active pharmaceutical ingredient (API). Suitable APIs include but are not limited to alpha-2 adrenergic agonists, analgesics, anesthetics, antibiotics (including agents having antimicrobial, antibacterial, antimycotic and/or antiprotozoal activity), antidepressants, antihistamines, antipsychotics, antivascular agents, antiviral agents, aptamers, artificial tears, beta-adrenergic blocking agents, carbonic anhydrase inhibitors, catalytic antioxidants, chemotherapeutics, cholinesterase inhibitors, corticosteroids direct acting miotics, hormones, light-activated drugs, non-steroidal anti-inflammatory drugs, ocular lubricants, ophthalmic decongestant agents, ophthalmic antiseptics, ophthalmic antifungals, peptides, prostaglandin analogs, proteins, catalytic antioxidants), sedatives, steroid, stimulants, sulfonamides, vasoconstrictors and vasodilators.

In embodiments of the present invention, the active ingredient is a peptides or proteins. Suitable peptides or proteins includde ACTH, angiotensin converting enzyme, bertilimumab, bevacizumab, calcitonin, concanavalin, dynorphin A, dynorphin B, enkephalins, endorphins, endothelin-1, enzyme, glial cell-line derived neurotrophic factor (GDNF), glucagon, gonadotropin releasing hormone, growth hormone rleasing hormone, hyaluronidase, ierdelimumab, IgGl, insulin, leptin, lerdelimumab, leucin-enkephalin, luterinizing hormone releasing hormone, lypressin, lysozyme, metelimumab, methionin-enkephalin, monoclonal antibodies, alpha-neoendorphine, beta-neoendorphin, neurotrophic factors, obestatin, oxytocin, peptide hormones, protein hormones, ranibizumab, ribonuclease, secretin, somatostatin, somatotropin, thyrotrophin releasing hormone, vasopressin, viral vectors and homologues thereof. In a preferred embodiment of the present invention, the active ingredient is selected from the group consisting of leptin and and homologues thereof. In a preferred embodiment of the present invention, the active ingredient is selected from the group consisting of antibodies or antibody homologues, such as IgGl.

In embodiments of the present invention, the peptide or protein active ingredient is a denaturizable active ingredient.

In embodiments of the present invention, the peptide or protein active ingredient has a molecular weight of greater than 1 kDa, greater than 1.5 kDa, greater than 3 kDa, greater than 5 kDa, greater than 10 kDa and even greater than 15 kDa.

In embodiments of the present invention, a composition further comprises a component selected from the group consisting of bioadhesives, buffering agents, chelating agents, humectants, pH-adjusting agents, preservatives, solubilizers, viscosity modifiers and vitamins.

According to the teachings of the present invention there is also provided a device for ophthalmic acministration of a composition, comprising: a) a misting unit including i) a nebulizer, configured to generate a mist from a composition; and ii) a mist director, configured to direct mist generated by the nebulizer at an eye; b) an eye-state detector, configured to detect if the eye is open or shut; and c) a switch functionally associated with the misting unit and with the eye-state detector having at least two states, an “ON” state wherein a mist is directed at the eye and an “OFF” state wherein a mist is not directed at the eye.

In embodiments of the present invention, the composition is a pharmaceutical composition.

In embodiments of the present invention the switch sets to the “ON” state when the eye-state detector detects that the eye is open.

In embodiments of the present invention the switch sets to the “OFF” state when the eye-state detector detects that the eye is shut.

In embodiments of the present invention the nebulizer is deactivated when the switch is set to the “OFF” state and the nebulizer is activated when the switch is set to the “ON” state.

In embodiments of the present invention, misting unit further comprises a blower (e.g., a fan or compressor) functionally associated with the mist director, the blower being deactivated when the switch is set to the “OFF” state and the blower being activated when the switch is set to the “ON” state.

In embodiments, the misting unit further comprises a valve functionally associated with the mist director, the valve configured to close when the switch is set to the “OFF” state and the valve configured to open when the switch is set to the “ON” state.

In embodiments, the eye state detector is configured to detect light reflecting from the surface of an anterior portion of an open eye.

According to the teachings of the present invention there is also provided a device for ophthalmic administ5ration of a pharmaceutical composition to an eye of a subject, comprising: a) a contact component with a contact surface, the contact surface configured to contact a portion of the body of the subject during the administration; and b) a reversibly actuatable radiation-source, configured to irradiate the contact surfface with sterilizing radiation.

In embodiments of the present invention the sterilizing radiation comprises radiation selected from the group consisting of microwave radiation, infrared radiation and ultraviolet radiation. In embodiments of the present invention the sterilizing radiation is selected from the group consisting of coherent radiation and incoherent radiation.

In embodiments of the present invention, the contact component and the radiation source are both integral elements of a single unit of the device.

In embodiments of the present invention, the single unit includes a power source (e.g., a battery, a fuel cell)

In embodiments of the present invention, the contact component is an integral element of a first unit of the device and the radiation source is an integral element of a second unit of the device, wherein the first unit and the second unit are physically distinct. In embodiments, the first unit includes a power source, preferably a rechargeable power source.

In embodiments of the present invention, the second unit includes a recharger for the power source.

In embodiments of the present invention, the sterilizing radiation is projected at the contact surface.

In embodiments of the present invention, the contact component acts as a wave guide to the sterilizing radiation.

In embodiments, the radiation-source is user-actuated. In embodiments, the radiation-source is automatically actuated. In embodiments, the radiation-source is autonomously actuated.

In embodiments, the device further comprises a fail-safe switch to prevent activation of the radiation source when the contact surface is in contact with the portion of the body.

According to the teachings of the present invention there is also provided a method of treatment, comprising: a) contacting a composition with a posterior section of an eye; b) shutting the eye with a respective eyelid; and c) vibrating the eyelid.

In embodiments of the present invention, the composition is a pharmaceutical composition.

in embodiments of the present invention, the contacting comprisese instilling a drop of the composition in the eye.

In embodiments of the present invention, the contacting comprises spraying the composition in the eye.

In embodiments of the present invention, the contacting comprises: i) generating a mist of the composition; and ii) contacting the mist with the cornea.

In embodiments of the present invention, vibrating the eyelid comprises contacting the eyelid with a vibrating physical component.

In embodiments of the present invention, the vibrating comprises vibrating at ultrasonic frequencies. In embodiments of the present invention, the vibrating comprises vibrating at sonic frequencies. In embodiments of the present invention, the frequencies comprise frequencies of between about 10 Hz and 100 mHz. In embodiments of the present invention, the frequencies comprise frequencies of no less than about 1 kHz, no less than about 10 kHz and even no less than about 1 mHz.

In embodiments of the present invention, the vibrating is for at least 10 seconds, at least 30 seconds and even for at least 60 seconds.

According to the teachings of the present invention there is also provided a device for increasing the bioavailability of an ophthalmically administered API in a pharmaceutical composition, comprising: a) an eyelid contact component, configured to physically contact an eyelid of an eye and maintain the eyelid in a shut position; and b) a vibration generator configured to generate vibrations (e.g., of ultrasonic and/or sonic frequencies) and transfer the vibrations to the eyelid contact component.

In embodiments of the present invention, the device further comprises a holder (e.g., a head band), configured to hold the eyelid contact component (e.g., an eye patch) against the eyelid.

In embodiments of the present invention, the vibration generator includes a piezoelectric crystal and/or a vibrating diaphragm.

In embodiments of the present invention, the vibration generator includes a liquid, an elastic material or the like to effectively transfer vibrations to the eyelid.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention. The description taken with the drawings making apparent to those skilled in the art how the several forms of the invention may be embodied in practice.

In the drawings:

FIGS. 1A-1H schematically depict an embodiment of a device of the present invention for administering a pharmaceutical composition, in accordance with the present invention.

FIGS. 2A-2E schematically depict an embodiment of a device of the present invention with a cradle for administering a pharmaceutical composition, in accordance with the present invention;

FIGS. 3A-3E schematically depict embodiments of computerized devices which may be used with a device of the present invention in accordance with the present invention;

FIG. 4 is a flowchart of an embodiment of a method of using an embodiment of a device of the present invention;

FIG. 5 schematically depicts a self-sterilizing ophthalmic delivery device of the present invention;

FIGS. 6A-B schematically depicts an eyelid-vibrating device of the present invention;

FIGS. 7A-7C display comparative results of delivery of a pharmaceutical composition including leptin to the retina and aqueous humor by instillation and as a mist;

FIGS. 8A-8B display comparative results of delivery of a pharmaceutical composition including leptin to the cerebrospinal fluid by instaillation and as a mist;

FIGS. 9A-9B display results of duration of delivery of a pharmaceutical composition including leptin to the sclera as a mist;

FIGS. 10A-10C display comparative results of delivery of a pharmaceutical composition including leptin to the retina, sclera and optic nerve by instillation and as a mist;

FIGS. 11A-11C display comparative results of delivery of a pharmaceutical composition including leptin to the serum by instillation and as a mist;

FIG. 12A is a reproduction of a photograph of a rat into which eye a pharmaceutical composition including saponin was instilled;

FIG. 12B is a reproduction of a photograph of a rat into which eye a pharmaceutical composition including saponin was administered as a mist; and

FIG. 13 displays results of mouse IgGl levels in the optic nerves of rats into which eye a pharmaceutical composition including mouse IgGl was administered as a mist.

### DESCRIPTION OF EMBODIMENTS OF THE INVENTION

The presewnt invention is related to the administration of compositions, especially of pharmaceutical compositions including an API into the eye by nebulizing a composition and then contacting the produced mist with an exposed anterior ophthalmic surface, such as the conjuncctiva, sclera or cornea.

An aspect of the present invention relates to the use of a mist of a pharmaceutical composition for ophthalmic administration of a peptide or protein active pharmaceutical ingredient, especially for delivery to the nervous system and the eye.

An additional aspect of the present invention relates to the use of a mist of a pharmaceutical composition for ophthalmic administration including a highly irritating penetration enhancer, whether inherently highly irritating or highly irritating at relatively high concentrations in a pharmaceutical composition.

An additional aspect of the present invention relates to the use of a mist of a pharmaceutical composition for ophthalmic administration of an API for selective delivery to the nervous system and the eye.

An additional aspect of the present invention relates to a device for ophthalmic administration comprising a nebulizer, a mist director to direct a generated mist at an eye, an eye-state detector to detect if an eye is open or closed, and a switch associated with both the eye-state detector and the mist director to direct mist at the eye only when open.

An additional aspect of the present invention relates to a device for ophthalmic administration that is self-sterilizing.

An additional aspect of the present invention is a method and a device for increasing the availability of an ophthalmically administered API, whether systemic or local, whether through the conjunctiva, sclera, cornea or other route, by vibrating the eyelid subsequent to the ophthalmic administration.

The principles, uses and implementations of the teachings of the present invention may be better understood with reference to the accompanying description, figures and examples. Upon perusal of the description and figures present herein, one skilled in the art is able to implement the teachings of the present invention without undue effort or experimentation. In the figures, like reference numerals refer to like parts throughout.

Befor explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of medicine, biology, chemistry, material sciences, pharmacology, and engineering. Such techniques are thoroughly explained in the literature.

Unless otherwise defined, all technical and scientific terms used herein have the same meaning as commonly understood by one of ordinary skill in the art to which the invention belongs. In addition, the descriptions, materials, methods and examples are illustrative only and not intended to be limiting. Methods and materials similar or equivalent to those described herein can be used in the practice or testing of the present invention.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the additon of one or more additional features, integers, steps, components or groups thereof but only if the additional features, integers, steps components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or radily developed from known manners, means, techniques and procedures by practitioners of the relevant arts. Implementation of the methods of the present invention involves performing or completing selected tasks or steps manually, automatically, or a combination thereof.

Herein, the term “active pharmaceutical ingredient” or API is understood to include a chemical, biological or pharmaceutical entities including any natural or synthetic chemical or biological substance that has a pharmaceutical effect. Typical APIs include but are not limited to antibodies, antigens, biological materials, chemical materials, drugs, enzymes, hormones, immunogenes, probes, tracers, nucleic acids, peptides, proteins, selective toxins and toxins.

Herein, the term “peptide” or “protein” is understood to include any polymer (dipeptide or greater) of amino acids (R or L, natural or not-natural) linked through peptide bonds. Thus, the terms include proteins, oligopeptides, protein fragments, analogs, muteins, fusion proteins and the like. These terms also encompass amino acid polymers as described above that include additional moieties such as glycoproteins, lipoproteins, phosphoproteins, metalloproteins, nucleoproteins, as well as other conjugated proteins. Herein, “peptide” refers to a polymer consisting of up to 40 amino acid residues whereas “protein” refers to a polymer consisting of more than 40 amino acid residues.

Herein, the term “penetration enhancer” is understood to include an component of a composition that increases the amount of absorption into the body of a substance coadministered therewith.

Herein, the term “ophtbalmically acceptable carrier” describes a carrier that does not cause significan irritation to the eye of an organism when applied in accordance with the teachings of the present invention and does not abrogate the pharmacological activity and properties of an API carried therewith.

Herein, the term “nebulizer” is understood to mean a device or a part of a device that converts a substance, e.g., a solid, gel, liquid, solution, suspension, ointment, pharmaceutical composition, into a mist.

Herein, the term “mist” refers to a cloud of particles having a mean partical diameter of less than about 20 microns, less than about 10 microns, less than about 8 microns, less than about 5 microns, less than about 3 micron and even less than about 1 micron.

An aspect of the present invention relates to the use of a mist of a pharmaceutical composition for ophthalmic delivery of a peptide or protein API to a subject (human or non-human) in need thereof. In embodiments, delivery is to the blood stream of the subject. In embodiments, delivery is selective to part of an eye (e.g., sclera, optic nerve and retina) or a part of the nervous system (e.g., the brain, the central nervous system, the cerebral cavity, the cerebrospinal fluid, and the spinal cord). The teachings of the present invention also provide a method of treatment where a pharmaceutical composition including a peptide or protein API and an ophthalmically acceptable carrier is provided, the composition nebulized; and the resulting mist contacted with a posterior surface of an eye of a subject in need thereof thereby depositing an effective amount of the API on the posterior surface. The teachings of the present invention also provide a device for ophthalmic administation of a pharmaceutical composition, comprising: a) a nebulizer, a composition reservoir functionally associated with the nebulizer; and within the reservoir a pharmaceutical composition including a peptide or protein API and an ophthalmically acceptable carrier.

An aspect of the present invention relates to the use of a mist for ophthalmic delivery of a pharmaceutical composition including a highly irritating penetration enhancer and an ophthalmically acceptable carrier (and preferably an API) to a subject (human or non-human) in need thereof. In embodiments, delivery is to the blood stream of the subject. In embodiments, delivery is selective to part of an eye (e.g., sclera, optic nerve and retina) or a part of the nervous system (e.g., the brain, the central nervous system, the cerebral cavity, the cerebrospinal fluid, and the spinal cord). The teachings of the present invention also provide a method of treatment where a pharmaceutical composition including a highly irritating penetration enhancer and an ophthalmically acceptable carrier (and preferably an API) is provided, the composition nebulized; and the resulting mist contacted with a posterior surface of an eye of a subject in need thereof. The teachings of the present invention also provide a device for ophthalmic administration of a pharmaceutical composition, comprising: a) a nebulizer; a composition reservoir functionally associated with the nebulizer; and within the reservoir a pharmaceutical composition including a highly irritating penetration enhancer and an ophthalmically acceptable carrier (and preferably an API).

An aspect of tyhe present invention relates to the use of a mist for ophthalmic delivery of a pharmaceutical composition for selective delivery to part of an eye (e.g., sclera, optic nerve and retina) or a part of the nervous system (e.g., the brain, the central nervous system, the cerebral cavity, the cerebrospinal fluid, and the spinal cord) of a subject (human or non-human) in need thereof. The teachings of the present invention also provide a method of treatment where a pharmaceutical composition including an API effective for treating a part of the eye or a part of the nervous system and an ophthalmically acceptable carrier, the composition nebulized; and the resulting mist contacted with a posterior surface of an eye of a subject in need thereof. The teachings of the present invention also provide a device for ophthalmic administration of a pharmaceutical composition, comprising: a) a nebulizer; a composition reservoir functionally associated with the nebulizer; and within the reservoir a pharmaceutical composition including an API effective for treating a part of the eye or a part of the nervous system and ophthalmically acceptable carrier.

The teachings of the present invention are generally applied for or in the context of treating the need of a subject. Typical needs include curing a condition, treating a condition, preventing a condition, treating symptoms of a condition, curing symptoms of a condition, ameliorating symptoms of a condition, treating effects of a condition, ameliorating effects of a condition, and preventing results of a condition. Typical conditions include but are not limited to behavioral conditions, brain disorders, cancer, eye cancers, brain cancers, cerebral cancers, nerve cancers, central nervous system disorders, choroidal neovascularization (such as associated with retinal or subretinal disorders, such as, age-related macular degeneration, presumed ocular histoplasmosis syndrome, myopic degeneration, angioid streaks and ocular trauma), corneal neovascularization (such as associated with trauma, chemical burns or corneal transplantation), glaucoma, infections, inflammatory diseases, inflammations, inflammatory diseases of the retina, intravitreal neovascularization (such as associated with diabetic retinopathy, vein occlusion, sickle cell retinopathy, retinopathy of prematurity, retinal detachment, ocular ischemia or trauma), iris neovacularization (such as associated with diabetic retinopathy, vein occlusion, ocular tumor or retinal detachment), macular edema, mental illnesses, neural conditions, neurological disorders, ocular diseases, ocular inflammation, optic disc neovascularization, optical nerve disorders, pannus posterior segment edema, postoperative ocular pain, proliferative vitreoretinopathy, prostaglandin formation, psychological conditions, psychoses and psychiatric disorders, pterygium, retinoblastoma, retinal edema, retinal degeneration, retinal revascularization (such as associated with diabetic retinopathy, vein occlusion, sickle cell retinopathy, retinopathy of prematurity, retinal detachment, ocular ischemia or trauma), uveitis and vascular retinopathy. In embodiments of the present invention, conditions are conditions susceptible to an interaction of an API with a nerve and/or with part of an eye (e.g., cornea, retina, vitreous fluid, sclera, lens).

In embodiments of the present invention, conditions are conditions susceptible to treatment with leptin or leptin homologues.

In embodiments of the present invention, conditions are conditions susceptible to treatment with antibodies or antibody homologues, such as IgGl.

In embodiments of the present invention, conditions are conditions suceptible to treatment with aptamers, such as anti-VEGF aptamers such as EYE01.

In embodiments of the present invention, the need requires delivery of an active ingredient to the blood stream and/or part of an eye (e.g., cornea retina, vitreous fluid, sclera, lens) and/or part of the nervous system (e.g., brain, the central nervous system, the cerebral cavity, the cerebrospinal fluid, an optic nerve, the retina and the spinal cord) of a subject.

Embodiments of the present invention are based on and supported by experiments performed and detailed below.

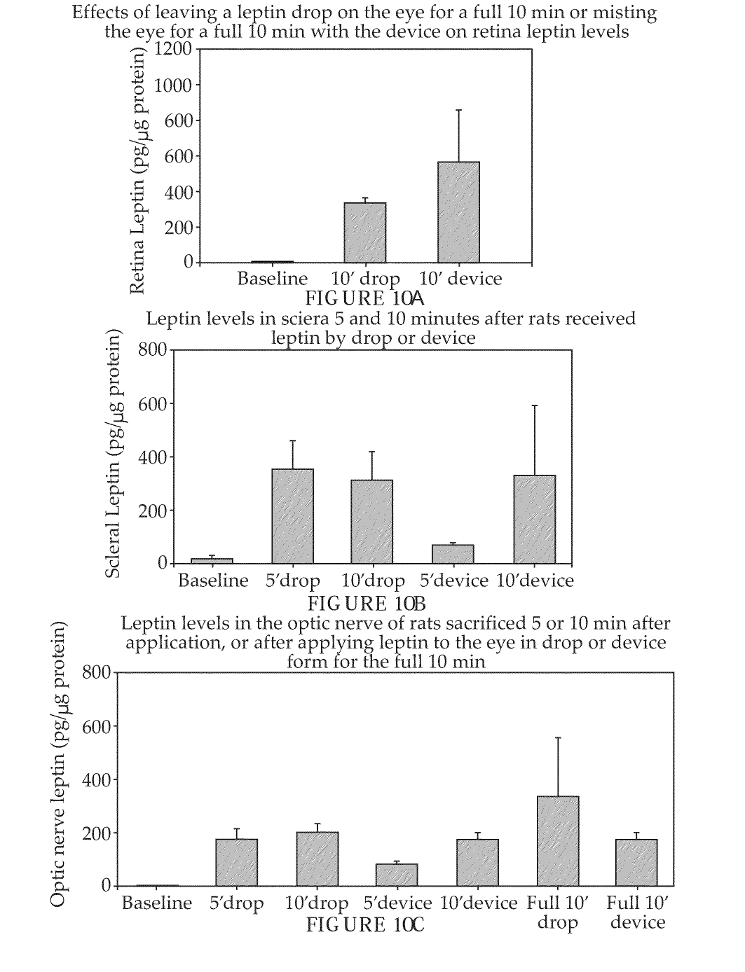

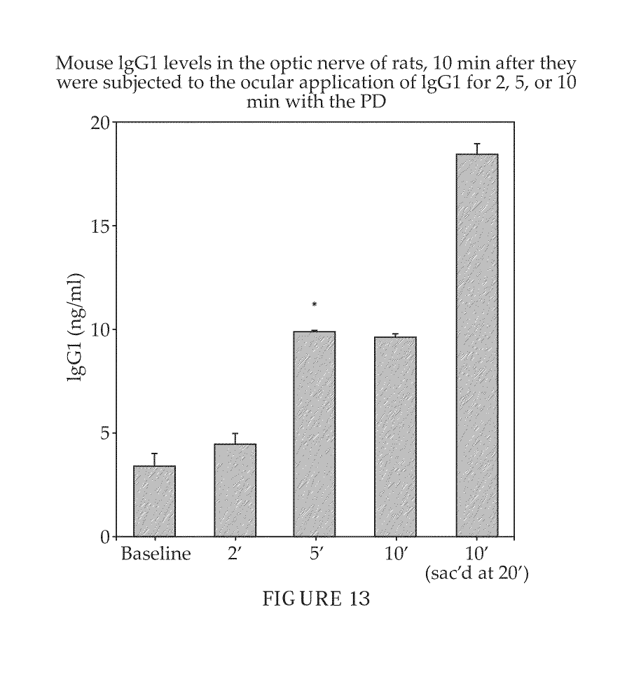

The experiments performed demonstrate that administration of a pharmaceutical composition including medium-sized proteins such as leptin or large sized proteins such as the antibody leptin as a mist allows effective delivery of an API to the back of the eye (optic nerve), see Experimental section FIGS. 10C and 13.

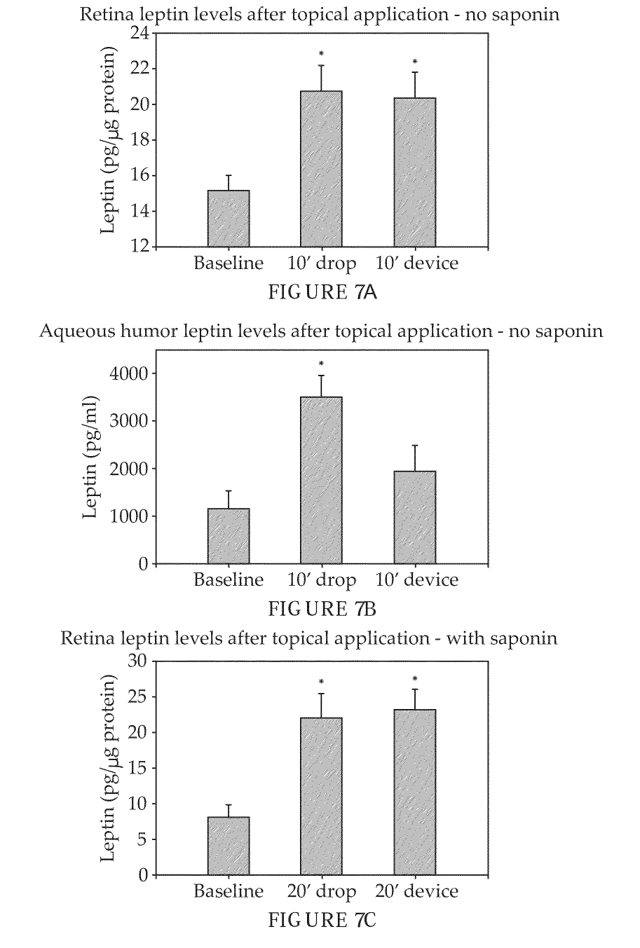

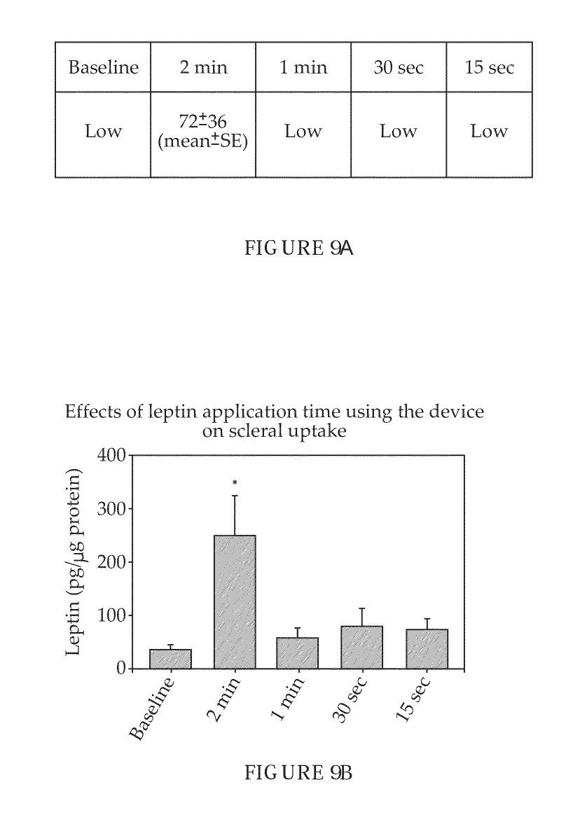

The experiments performed demonstrate that an API administered ophthalmically in a nebulized pharmaceutical composition is transported from the posterior section of the eye by a mechanism different than when administered by instillation, see Experimental section FIGS. 7B, 9A and 9B.

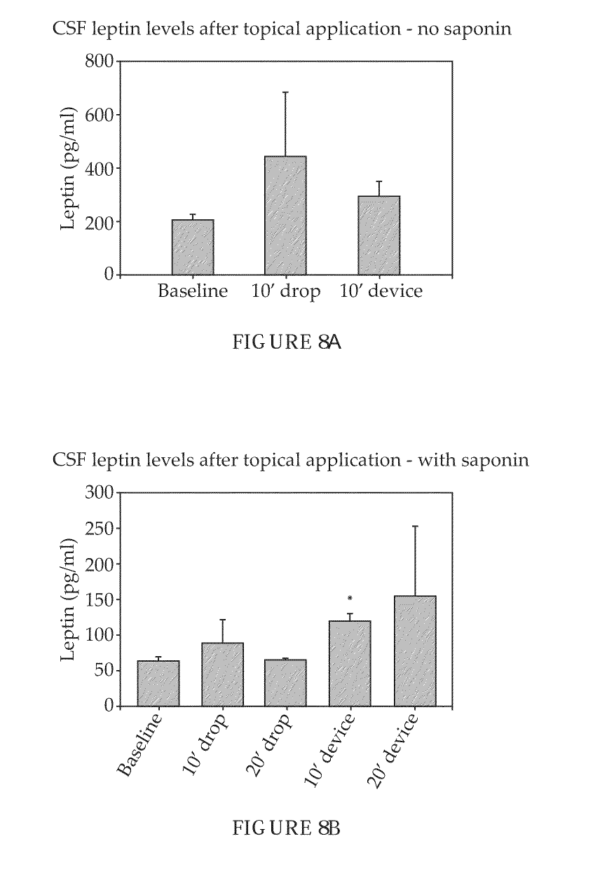

The experiments performed demonstrate that administration of a pharmaceutical composition as a mist, but not by instillation, allows delivery of an API to the cerebrospinal fluid (CSF), see Experimental section FIG. 8B.

The experiments performed demonstrate that administration of a pharmaceutical composition as a mist in accordance with embodiments of the present invention allows the use of otherwise irritant penetration enhancers, see Experimental section FIGS. 12A and 12B.

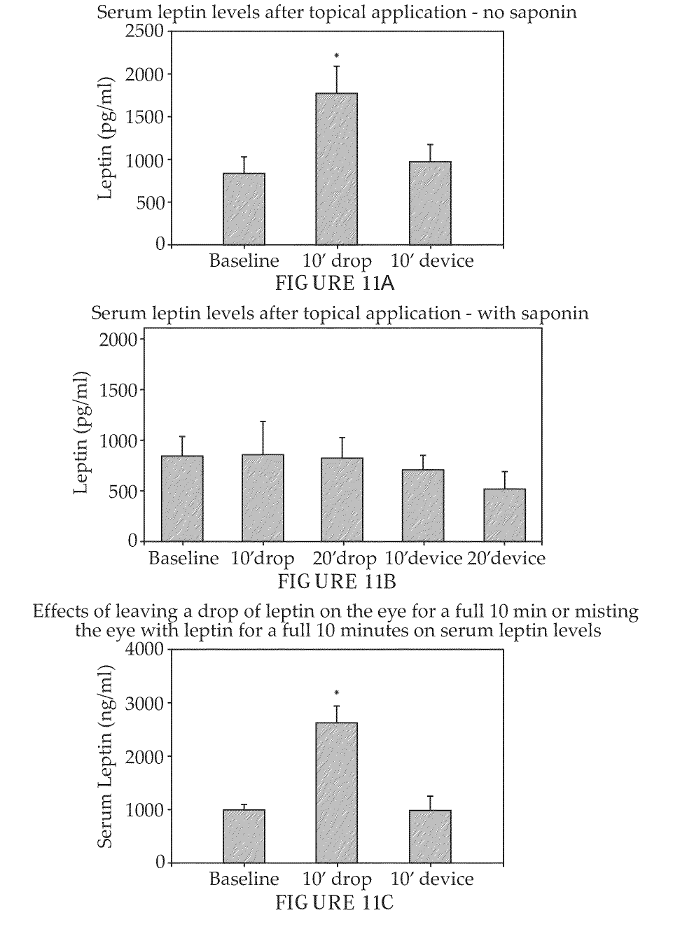

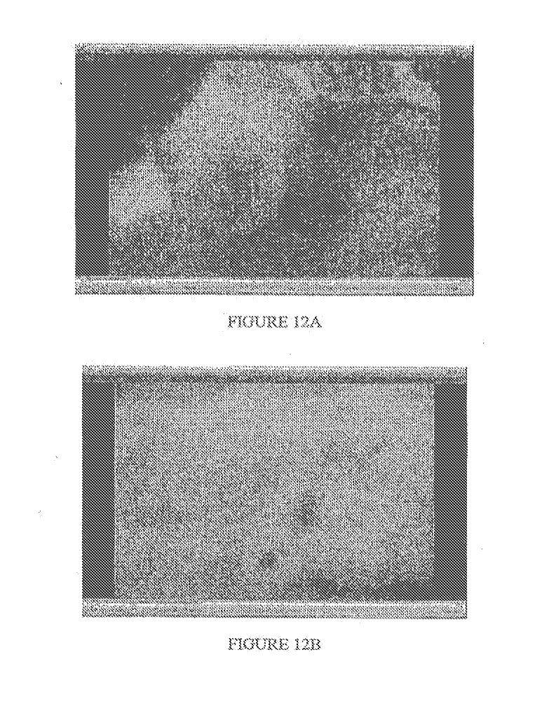

The experiments performed demonstrate that administration of a pharmaceutical composition including a penetration enhancer as a mist in accordance with embodiments of the present invention is relatively selective, delivering higher or comparable amounts of API to the retina, sclera, optic nerve and central nervous system (CNS), see Experimental section FIGS. 7A, 7C, 8A, 8B, 10A, 10B, 10C, but delivering significantly less API to the serum and to the aqueous humor than when administered by instillation, see Experimental section FIGS. 7B, 11A, 11C.

It is important to note, that the experiments detailed below on which the above conclusions and invention are based, compare ophthalmic administration of a composition as a mist to administration by instillation. These experiments indicate that delivery of an API to the retina, sclera and optic nerve by either method provides similar concentrations of API in the tissue. However, as in the experiments instillation included forcibly shutting the eye of a subject rat for two minutes, it is expected that in actual clinical use, administration of a composition using a mist in accordance with the teachings of the present invention will be more effective than instillation of the same composition.

In the art, the systemic delivery of small peptide or protein APIs such as insulin is well established by instillation of a pharmaceutical composition including the API. Instillation is considered the preferred method of ophthalmic administration as the large volume of liquid instilled overcomes the ocular protective mechanisms. The evidence indicates that the API is washed to the conjunctiva and there absorbed through the mucosa. Once in the blood stream, such an API acts systemically and cannot reach certain organs, for example the central nervous system. Further, as many peptide and protein APIs have very subtle effects, systemic administration may be contraindicated.

An aspect of the present invention relates to the use of a mist of a pharmaceutical composition for ophthalmic delivery of a peptide or protein active pharmaceutical ingredient, especially for delivery to the eye and nervous system.

The experimental data presented below demonstrates that the teachings of the present invention allow for advantageous ophthalmic delivery of peptides and proteins.

Embodiments of the teachings of the present invention provide for the delivery of denaturable peptides and protein APIs that do not lose an active tertiary or quaternary structure including APIs having a molecular weight of greater than 1 kDa, greater than 1.5 kDa, greater than 3 kDa, greater than 5 kDa, greater than 10 kDa and even greater than 15 kDa.

Embodiments of the teachings of the present invention provide for the delivery of peptide and protein APIs, systemically (e.g., to the blood stream) or selectively.

Embodiments of the teachings of the present invention provide for the selective delivery of peptide and protein APIs, especially selective delivery to the eye (e.g., sclera, optic nerve and retina) and/or the nervous system (the brain, the central nervous system, the cerebral cavity, the cerebrospinal fluid, and the spinal cord).

Administration of a peptide or protein API in accordance with the teachings of the present invention is to a subject (human or non-human) in need thereof. Typical needs include curing a condition, treating a condition, preventing a condition, treating symptoms of a condition, curing symptoms of a condition, ameliorating effects of a condition, and preventing results of a condition. Typical conditions include, but are not limied to behavioral conditions, brain disorders, cancer, eye cancers, brain cancers, cerebral cancers, nerve cancers, central nervous system disorders, choroidal neovascularization (such as associated with retinal or subretinal disorders, such as, age-related macular degeneration, presumed ocular histoplasmosis syndrome, myopic degeneration, angioid streaks and ocular trauma), corneal neovascularization (such as associated with trauma, chemical burns or corneal transplantation), glaucoma, infections, inflammatory diseases, inflammations, inflammatory diseases of the retina, intravitreal neovascularization (such as associated with diaebetic retinopathy, vein occulsion, sickle cell retinopathy, retinopathy of prematurity, retinal detachment, ocular ischemia or trauma), iris neovascularization (such as associated with diabetic retinopathy, vein occlusion, ocular tumor or retinal detachment), macular edema, mental illnesses, neural coditions, neurological disorders, ocular diseases, ocular inflammation, optic disc neovascularization, optical nerve disorders, pannus posterior segment edema, postoperative ocular pain, proliferative vitreoretinopathy, prostaglandin formation, psychological conditions, psychoses and psychiatric disorders, pterygium, retinoblastoma, retinal edema, retinal degeneration, retinal revascularization (such as assocciated with diabetic retinopathy, vein occjusion, sickle cell retinopathy, retinopathy of prematurity, retinal detachment, ocular ischemia or trauma), uveitis and vascular retinopathy.

Peptide and protein APIs that are advantageously delivered in accordance with the teachings of the present invention include but are not limited to ACTH, angiotensin converting enzyme, bertilimumab, bevacizumab, calcitonin, concanavalin, dynorphin A, dynorphin B, endothelin-1, enkephalins, endorphins, enzyme, glial cell-line derived neurotrophic factor (GDNF), glucagon, gonadotropin releasing hormone, growth hormone releasing hormone, hyaluronidase, ierdelimumab, IgGl, insulin, leptin, lerdelimumab, leucine-enkephalin, luteinizing hormone releasing hormone, lypressin, lysozyme, metelimurnab, methionine-enkephalin, monoclonal antibodies, alpha-neoendorphin, beta-neoendorphin, neurotrophic factors, obestatin, oxytocin, peptide hormones, protein hormones, ranibizumab, ribonuclease, secretin, somatostatin, somatotropin, thyrotrophin releasing hormone, vasopressin, viral vectors and homologues thereof.

A preferred protein delivered in accordance with the teachings of the present invention is leptin or leptin homologues to treat conditions susceptible to treatment with leptin or leptin homologues such as weight control (Zhang, Y. et al. Nature 1994, 372, 425-432), female sexual development and hormonal imbalance, immune system disorders, bone development and retinal neuron preservation (Koeberle, P D et al. Vision Res. 1998, 38, 1505-1515 and Schmeer, C. et al. Eur. J. Neurosci. 2002, 15, 637-643), for example during diabetic retinopathy, macular degeneration, retinitis pigmentosa.

A preferred type of protein delivered in accordance with the teachings of the present invention are antibodies and antibody homologues, especially IgGl to treat conditions susceptible to treatment with antibody and antibody homologues, for example to immunize populations or to inactivate factors, such as VEGF, that induce vascular leakage and neovascularization in the retina in various diseases such as wet age-related macular degeneration. Further, in the experimental section it is demonstrated that IgGl administered in accordance with the teachings of the present invention accumulates in the optic nerve. Since the optic nerve is surrounded by CSF and is directly connected to the brain, it is expected that extremely large proteins such as antibodies such as IgGl are deliverable to the central nervous system using the teachings of the present invention as demonstrated for leptin.

Penetration enhancers are materials that transiently increase the permeability of the corneal epithelium or conjunctiva to facilitate API penetration therethrough. The use of known percutaneous penetration enhancers has been proposed (see Sasaki et al. Crit. Rev. Ther. Drug Carrier Syst. 1999, 16, 85-146) but is not generally used due to observations of irritation and corneal and conjunctival injury caused by known penetration enhancers, see Saettone et al. Int. J. Pharm. 1996, 142, 103-113 and Furrer et al. AAPS Pharm. Sci. 2002, 4(1), 1-5)

Penetration enhancers can be classified as being inherently highly irritating to the eye and as being mildly irritating to the eye.

Although inherently highly irritating penetration enhancers are expected to be more effective at enhancing the penetration and consequently bioavailability of APIs in ophthalmically administered pharmaceutical compositions, such inherently highly irritating penetration enhancers are not used due to the danger of grievous injury and even blindness to a subject to to which such compositions are applied.

As a result, in the art it is known to use only mildly irritating penetration enhancers in ophthalmically administered pharmaceutical compositions. However, as mildly irritating penetration enhancers do cause discomfort and may damage the eye, the concentration of mildly irritating penetration enhancers in known ophthalmically pharmaceutical compositions is limited to a relatively low and ineffective level.

An additional aspect of the present invention relates to the use of a mist of a pharmaceutical composition for ophthalmic delivery of a including a highly irritating penetration enhancer, whether inherently highly irritating or highly irritating at relatively high concentrations in a pharmaceutical composition.

Inherently highly irritating penetration enhancers useful for implementing the teachings of the present invention, for example as components of an embodiment of a composition of the present invention include, but are not limited to benzalkonium chloride, BL-9, deoxycholic acid, digitonin, escin, fusidic acid, fusidate, fusidic acid derivatives, saponin, saponins, sodium deoxycholate, acetone, acyl lactylates, acyl peptides, acylsarcosinates, alcohols, alkanolamine salts of fatty acids, alkyl benzene sulphonates, alkyl ether sulphates, alkyl sulphates, allantoin, anionic surface-active agents, 1-substituted azacycloheptan-2-ones, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dioctyl adipate, dioctyl azelate, dioctyl sebacate, 1,4-dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, glycerol monolaurate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, lecithin, maypons, metal salts of fatty acids, methyl nicotinate 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyethylene glycol monolaurate, polyglycerol esters, poly(vinyl pyridinium chloride), propan-1-ol, propan-2-ol, propylene glycol, propylene glycol dipelargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sorbitan monooleate, sorbitan monolaurate, sugar esters, sulphosuccinate, tetrahydrofuran, tetrahydrofurfural alcohol, transcutol, triethanolamine dodecyl benzene sulphonate, triethanolamine oleate, urazole, urea and derivatives, esters, salts and mixtures thereof. In embodiments of the present invention, an inherently highly irritating penetration enhancer comprises at least 0.05%, at least 0.1%, at least 0.2%, at least 0.5%, at least 1% and even at least 2% by weight of the pharmaceutical composition.

A preferred inherently highly irritating penetration enhancer useful for implementing the teachings of the present invention is saponin.

Penetration enhancers that are highly irritating at high concentrations useful for implementing the teachings of the present invention, for example as components of an embodiment of a composition of the present invention include, but are not limited to ammonium glycyrrhizide, Brij 35, Brij 78, Brij-98, cetylpyridium chloride, chenodeoxycholic acid, cholate, cholic acid, decamethonium, decamethonium bromide, dimethyl sulphoxide, EDTA and disodium EDTA, glycocholate, glycocholic acid, glycodeoxycholic acid, glycyrrhizic acid, paraben, polyoxyethylene, polyoxyethylene ethers of fatty acids such as polyoxyethylene 4-, 9-, 10-, and 23-lauryl ether, polyoxyethylene 10- and 20 -cetyl ether, polyoxyethylene 10- and 20-stearyl ether, polyoxyethylated castor oil, polyoxyethylene monolaurate, polyoxyethylene sorbitans such as polyoxyethylene sorbitan monolaurate, polyoxy:polyoxyethylene stearate, polyoxypropylene 15 stearyl ether, sodium cholate, sodium glycocholate, sodium taurocholate, sodium glycodeoxycholate, sodium taurodeoxycholate, sodium ursodeoxycholate, taurocholic acid, taurodeoxycholic acid, TWEEN 20, urosdeoxycholic acid, and derivatives, esters, salts and mixtures thereof in a greater than accepted concentration. In embodiments of the present invention, an inherently highly irritating penetration enhancer comprises at least 0.05%, at least 0.1%, at least 0.2%, at least 0.5%, at least 1%, at least 2%, at least 3% and even at least 4% by weight of the pharmaceutical composition.

Preferably, a composition of the present invention is a pharmaceutical composition including an API. As noted above, in embodiments a composition of the present invention includes a peptide or protein API. In embodiments, a composition of the present invention includes one or more non-peptide or protein APIs. In embodiments, a composition of the present invention includes one or more non-peptide or protein APIs in addition to a peptide or protein API. In embodiments, a composition of the present invention is devoid of a peptide or protein API.

Embodiments of the teachings of the present invention provide for the selective delivery of APIs, especially selective delivery to the eye (e.g., sclera, optic nerve and retina) and/or the nervous system (the brain, the central nervous system, the cerebral cavity, the cerebrospinal fluids, and the spinal cord). Administration of an API in accordance with the teachings of the present invention is to a subject (human or non-human) in need thereof. Typical needs include curing a condition, treating a condition, preventing a condition, treating symptoms of a condition, curing symptoms of a condition, ameliorating symptoms of a condition, treating effects of a condition, ameliorating effects of a condition, and preventing results of a condition. Typical conditions include, but are not limited to, behavioral conditions, brain disorders, cancer, eye cancers, brain cancers, cerebral cancers, nerve cancers, central nervous system disorders, choroidal neovascularization (such as associated with retinal or subretinal disorders, such as, age-related macular degernaetion, presumed ocular histoplasmosis syndrome, myopic degeneration, angioid streaks and ocular trauma), corneal neovascularization (such as associated with trauma, chemical burns or corneal transplantation), glaucoma, infections, inflammatory diseases, inflammations, inflammatory diseases of the retina, intravitreal neovascularization (such as associated with diabetic retinopathy, vein occlusion, sickle cell retinopathy, retinopathy of prematurity, retinal detachment, ocular ischemia or trauma), iris neovascularization (such as associated with diabetic retinopathy, vein occlusion, ocular tumor or retinal detachment), macular edema, mental illnesses, neural conditions, neurological disorders, ocular diseases, ocular inflammation, optic disc neovascularization, optical nerve disorders, pannus posterior segment edema, postoperative ocular pain, proliferative vitreoretinopathy, prostaglandin formation, psychological conditions, psychoses and psychiatric disorders, pterygium, retinoblastoma, retinal edema, retinal degeneration, retinal revascularization (such as associated with diabetic retinopathy, vein occlusion, sickle cell retinopathy, retinopathy of prematurity, retinal detachment, ocular ischemia or trauma), uveitis and vascular retinopathy.

Non-peptide and protein APIs that are advantageously delivered in accordance with the teachings of the present invention include but are not limited to alpha-2 adrenergic agonists, analgesics, anesthetics, antibiotics, antidepressants, antihistamines, antipsychotics, antivascular agents, antiviral agents, artificial tears, beta-adrenergic blocking agents, carbonic anhydrase inhibitors, catalytic antioxidants, chemotherapeutics, cholinesterase inhibitors, corticosteroids, direct acting miotics, hormones, light-activated drugs, non-steroidal, anti-inflammatory drugs, ocular lubricants, ophthalmic decongestant agents, ophthalmic antiseptics, ophthalmic antifungals, peptides, prostaglandin analogs, proteins, catalytic antioxidants), sedatives, steroid, stimulants, sulfonamides, vasoconstrictors.

In embodiments, a composition of the present invention includes an analgesic API. Suitable analgesics include, but are not limited to, benzocaine, butamben picrate, dibucaine, dimethisoquin, dyclonine, lidocaine, pramoxine, tetracaine, salicylates and derivatives, esters, salts and mixtures thereof.

In embodiments, a composition of the present invention inludes an anesthetic. Suitable anesthetics include, but are not limited to, benzocaine, bupivacaine, butamben picrate, chlorprocaine, cocaine, dibucaine, dimethisoquin, dyclonine, etidocaine, hexylcaine, ketamine, lidocaine, mepivacaine, pramoxine, procaine, tetracaine, salicylates and derivatives, ester, salts and mixtures thereof.

In embodiments, a composition of the present invention includes an aptamer. Suitable aptamers include anti-VEGF aptamers such as EYE01. In embodiments of the invention, aptamers are encapsulated for controlled release, for example within microsphere. A description of anti-VEGF aptamers such as EYE01 and the encapsulation hereof in microspheres is discussed in U.S. Patent Application 2005/0175708, incorporated by reference as if fully set forth herein.

In embodiments, a composition of of the present invention includes an antibiotic, including agents with antimicrobial, antibacterial, antimycotic and/or antiprotozoal activity. Suitable analgesics include, but are not limited to, amanfadine hydrochloride, amanfadine sulfate, amikacin, amikacin sulfate, aminoglycosides, amoxicillin, ampicillin, ansamycins, bacitracin, beta-lactams, butoconazole, candicidin, capreomycin, carbenicillin, cephalexin, cephaloridine, cephalothin, cefazolin cephapirin, cephradine, cephaloglycin chloramphenicols, chlorhexidine, chlorhexidine gluconate, chlorhexidine hydrochloride, chloroxine, chlorquinaldol, chlortetracycline, chlortetracycline hydrochloride, ciclopirox olamine, ciprofloxacin, circulin, clindamycin, clindamycin hydrochloride, clotrimazole, cloxacillin, demeclocycline, diclosxacillin, diiodohydroxyquin, doxycycline, econazole, elubiol, ethambutol, ethambutol hydrochloride, erythromycin, erythromycin estolate, erythomycin stearate, farnesol, floxacillin, fluconazole, gentamicin, gentamicin sulfate, gramicidin, griseofulvin, haloprogin, haloquinal, hexachlorophene, iminocycline, iodochlorhydroxyquin, itraconazole, kanamycin, kanamycin sulfate, ketoconazole, lincomycin, lincomycin, lincomycin hydrochloride, macrolides, mafenide acetate, meclocycline, methacycline, methacycline hydrochloride, methenamine, methenamine hippurate, methenamine mandelate, methicillin, metronidazole, miconazole, miconazole hydrochloride, minocycline, minocycline hydrochloride, mupirocin, nafcillin, neomycin neomycin sulfate, netilmicin, netilmicin sulfate, nitrofurazone, norfloxacin nystatin, octopirox, oleandomycin, orcephalosporins, oxacillin, oxiconazole, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, terbinafine, terconazole, tetracycline, tioconazole, tobramycin, tolnaftate, triclosan, trifampin, rifamycin, rolitetracycline, spectinomycin, spiramycin, streptomycin, sulconazole, sulfonamide, tetracyclines, tetracycline, tobramycin, tobramycin sulfate, triclocarbon, triclosan, timethoprim-sulfamethoxazole, tylosin, undecylenic acid, vancomycin yrothricin and derivatives, esters, salts and mixtures thereof.

In embodiments a composition of of the present invention includes an antidepressant. Suitable antidepressants include, but are not limited to, α-adrenoreceptor antagonists, corticotropin-releasing factor antagonists, monoamine-oxidase inhibitors, 5-HT_{1A}-receptor agonist antagonists, NK1-receptor antagonists, norepinephrinere-reuptake inhibitors, selective-serotonin-reuptake inhibitors, serotonin-and-noradrenaline-reuptake inhibitors, tetracyclic antidepressant, tricyclic antidepressant, amitriptyline, adinazolam, amiltriptylinoxide, amoxapine, amineptine, butriptyline, binedaline, biproprion hydrochloride, m-chloropiperzine, citalopram, clomipramine, demexiptiline, desipramine, desmethylamitriptyline, dibenzepin, dimetacrine, dothiepin doxepin, duloxetine, etoperidone, femoxetine, fluacizine, fluoxetine, fluvoxamine, imipramine, imipramine-oxide, indalpine, indeloxazine, iprindole, lofepramine, maprotiline, melitracen, metapramine, milnacipran, mitrazapine, nefazodone, norclolipramine, nortripytyline, noxiptilin, opipramol, oxaflazone, paroxetine, perlapine, pizotyline, prolintane, propizepine, protriptyline, quinupramine, reboxetine, ritanserin, sertraline, trimipramine, tianeptine, tandospirone, trazadone, venlafaxine, zimeldine and derivatives, esters, salts and mixtures thereof.

In embodiments, a composition of of the present invention includes an antihistamine. Suitable antihistamines include, but are not limited to, chlorcyclizine, diphenhydramine, mepyramine, methapyrilene, tripelennamine and derivatives, esters, salts and mixtures thereof.

In embodiments, a composition of of the present invention includes an antipsychotic. Suitable antipsychotics include, but are not limited to, selective serotonin-reuptake inhibitors, fluoxetine, fluvoxamine, sertraline, escitalopram, citalopram, paroxetine, monoamine oxidase inhibitors, isocarboxazid, phenelzine, tranylcypromine, conventional antipsychotics, haloperidol, molindone, thioridazine, atypical antipsychotics, clozapine, olanzapine, risperidone, quetiapine, sertindole, aripiprazole, ziprasidone, and derivatives, esters, salts and mixtures thereof.

In embodiments, a composition of of the present invention includes a corticosteroid. Suitable corticosteroids include, but are not limited to, alclometasone dipropionate, amcinafel, amcinafide, amcinonide, beclomethasone, beclomethasone dipropionate, betamethsone, betamethasone benzoate, betamethasone dexamethasone-phosphate, betamethasonedipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desocymethasone, dexamethasone, desoxycorticosterone acetate, dichlorisone, diflorasone diacetate, difluorcortolone valerate, diflurosone diacetate, difharprednate, fluadrenolone, flucetonide, flucinolone acetonide, flucloronide, fluclorolone acetonide, flucortine butylesters, fludroxycortide, fludrocortisone, flumethasone, flumethasone pivalate, flumethasone pivalate, flumisolide, fluocinolone, fluocinolone acetonide, fluocinonide, fluocortin butyl, fluocortolone, fluorometholone, fluosinolone acetonide, fluperolone fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, α-methyl dexamethasone, methylprednisolone, methylprednisolone acetate, mometasone furoate, paramethasone, prednisolone, prednisone, pregnenolone, progesterone, spironolactone, triamcinolone, traiamcinolone acetonide and derivatives, esters, salts and mixtures thereof.

In embodiments, a composition of of the present invention includes a hormone. Suitable hormones include, but are not limited to, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenopropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5a-pregnan-3b,20a-diol sulfate 5a-pregnan-3b,20b-diol sulfate, 5a-pregnan-3b-ol-20-one, 16,5a-prenen-3b-ol-20-one, 4-pregnen-20b-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and derivatives, esters, salts and mixtures thereof.

In embodiments, a composition of of the present invention includes a non-steroidal anti-inflammatory drug. Suitable non-steroidal anti-inflammatory drugs include, but are not limited to, acemaiacin, acetic acid derivatives, alminoprofen, amfenac, aspirin, azapropazone, azelaic acid, benorylate, benoxaprofen, carprofen, clindanac, CP-14,304, diclofenac, diflunisal, disalcid, felbinac, fenamates, fenbufen,fenclofenac, fendosal, fenoprofen, fentiazac, feprazone, flufenamic, flurbiprofen, furofenac, ibuprofen, indomethacin, indopropfen, isoxepac, isoxicam, ketoprofen, ketorolac, meclofenamic, mefenamic, miroprofen, naproxen, nepafenac, niflumic, oxaprozin, oxepinac, oxicams, oxyphenbutazone, phenylbutazone, piroxicam, pranoprofen, propionic acid derivatives, pirprofen, pyrazoles, safapryn, salicylates, solprin, sudoxicam, sulindac, suprofen, tenoxicam, tiopinac, tiaprofen, tioxaprofen, tolfenamic acids, tolmetin, trilisate, trimethazone, zidometacin, zomerpirac and derivatives, esters, salts and mixtures thereof.

In embodiments, a composition of of the present invention includes an alpha-2 adrenergic agonist. Suitable alpha-2 adrenergic agonists include, but are not limited to, apraclonidine, brimonidine tartrate, dapiprazole and dipivefrin.

In embodiments, a composition of of the present invention includes a antivascular agent. Suitable antivascular agents include, but are not limited to, anecortave acetate, pegaptanib and squalamine.

In embodiments, a composition of of the present invention includes beta-adrenergic blocking agent. Suitable beta-adrenergic blocking agents include, but are not limited to, betaxolol, carteolol, levobunolol, metipranolol and timolol maleate.

In embodiments, a composition of of the present invention includes a carbonic anhydrase inhibitors. Suitable carbonic anhydrase inhibitors include, but are not limited to, acetazolamide, brinzolamide, dorzolamide, methazolamide, neptzane and unoprostone isopropyl.

In embodiments, a composition of of the present invention includes a catalytic antioxidants. Suitable catalytic antioxidants include, but are not limited to, OT-551 and OT-730.

In embodiments, a composition of of the present invention includes a cholinesterase inhibitor. Suitable cholinesterase inhibitors include, but are not limited to, echothiopate iodide.

In embodiments, a composition of of the present invention includes a direct acting miotic. Suitable direct acting miotics include, but are not limited to, carbachol and pilocarpine.

In embodiments, a composition of of the present invention includes a light-activated drugs. Suitable light-activated drugs include, but are not limited to, tin ethyl etiopurpurin and verteporfin.

In embodiments, a composiition of of the present invention includes an ophthalmic decongestant agent. Suitable ophthalmic decongestant agent include, but are not limited to, iodoxamide tromethamine and tromethamine.

In embodiments, a composition of of the present invention includes a prostaglandin analog. Suitable prostaglandin analog include, but are not limited to, bimatoprost, latanoprost, travoprost and unoprostone.

In embodiments, a composition of of the present invention includes a vasodilator. Suitable vasodilators include, but are not limited to, isosorbide dinitrate and hesperidin.

In embodiments, a composition of of the present invention includes a vasoconstrictor. Suitable vasoconstrictors include, but are not limited to, naphazoline and phenylephrine.

Specific APIs that preferably constitute part of a composition of the present invention include, but are not limited to, Brimonidine tartrate, Dipivefrin HCl, Apraclonidine HCl, Dapiprazole, Dorzolamide, Timolol, Dorzolamide with Timolol, Unoprostone Isopropyl, Levobunolol, Betaxolol, Pilocarpine, Echothiphate Iodide, Latanoprost, Bimatoprost, Flurbiprofen Sodium, Prednisolone Acetate, Dexamethasone, Triamcinolone acetonide and Nepafenac.

### Brimonidine Tartrate

Brimonidine tartrate (5-bromo-6-(2-imidazolidinylideneamino) quinoxaline L-tartrate) is an alpha-2 adrenergic agonist known as an API for treating ocular hypertension in glaucoma sufferers commercially available as an instillable solution including 0.2% (2 mg/ml) or 0.15% (1.5 mg/ml) brimonidine tartrate as Aphagan® or Alphagan® P, respectively, both of Allergan Inc (Irvine, Calif., USA).

In an embodiment of the present invention, a composition is provided comprising an ophthalmic carrier, a highly irritant penetration enhancer (e.g., saponin fusidate, azone, bile acid salts such as glycholate and cholate) and brimonidine tartrate and is administered in accordance with the teachings of the present invention. the coadministration of brimonidine tartrate and a highly irritant penetration enhancer generally allows for increased bioavailability and more effective treatment of conditions for which treatment with brimonidine tartrate is useful including glaucoma and ocular hypertension.

The coadministration of brimonidine tartrate and a highly irritant penetration enhancer in accordance with the teachings of the present invention generally increases the bioavailability of the brimonidine tartrate, allowing for administration of a reduced dose of aPI. For example, a prior art course of treatment of ocular hypertension is one drop (approximately 39 microliter) of Alphagan® P per eye every eight hours. In contrast, in embodiments of the present invention treatment of ocular hypertension includes administering between about 1 microliter and about 39 microliters, preferably between 1 microliters and about 20 microliters of a composition including 0.15% brimonidine tartrate and a highly irritant penetration enhancer to each eye every eight hours. The composition is administered is a mist using a nebulizing device such as a device of the present invention.

### Dipivefrin HCl

Dipivefrin HCl ((±) 3, 4-dihydroxy-alpha-[(methylamino)methyl] benzyl alcohol 3,4-dipivalate hydrochloride) is a prodrug formed by the diesterification of epinephrine and pivalic acid. The addition of the pivaloyl groups to the epinephrine molecule enhances the molecules lipophilic character increasing penetration into the anterior chamber of the ye when topically applied. Dipivefin HCl is converted to epinephrine inside the human eye by enzyme hydrolysis. The liberated epinephrine, and adrenergic agonist, appears to exert its action by decreasing water production and by enhancing aqueous outflow. Dipivefrin HCl is commercially available as an instillable solution including 0.1% (1 mg/ml) dipivefrin HCl under the tradename Propine® of Alergan Inc (Irvine, Calif., USA).

In an embodiment of the present invention, a composition is provided comprising an ophthalmic carrier, a highly irritant penetration enhancer (e.g., saponin, fusidate, azone, bile acid salts such as glycholate and cholate) and dipivefrin HCl and is administered in accordance with the teachings of the present invention. The coadministration of dipivefrin HCl and a high irritant penetration enhancer in accordance with the teachings of the present invention generally increases the bioavailability of the dipivefrin HCl, allowing for administration of a reduced dose of API and more effective treatment of conditions for which treatment with dipivefrin HCl is useful, including glaucoma (especially chronic open-angle glaucoma) and ocular hypertension.

For example, a prior art course of treatment of ocular hypertension is one drop (approximately 39 microliter) of Propine® per eye every twelve hours. In contrast, in embodiments of the present invention treatment of ocular hypertension includes administering between about 1 microliter and about 39 microliters, preferably between 1 microliters and about 20 microliters of a composition including 0.1% dipivefrin HCl and a highly irritant penetration enhancer to each eye every twelve hours. the compositiion is administered is a mist using a nebulizing device such as a device of the present invention.

### Apraclonidine HCl

Apraclonidine HCl (2-[(4-amino-2,7 dichlorophenyl) imino]imidazolidine monohydrochloride) is a relatively selective alpha-2-agonist vasoconstrictor. Apraclonidine acts on adrenoceptors in the walls of blood vessels in the eye. This causes the blood vessels to narrow, restricting the flow of blood threrethrough. Reduced blood flow leads to a decrease in the production of the aqueous humour decreasing the pressure created within the eye by the fluid. Decreasing the pressure within the eye is important in the treatment of conditions such as glaucoma and prior to or after laser surgery on the eye. Apraclonidine HCl is commercially available in an instillable solution including 0.5% (5 mg/ml) Apraclonidine under the tradename Iopidine® of Alcon USA (Fort Worth, Tex., USA).

In an embodiment of the present invention, a composition is provided comprising an ophthalmic carrier, a highly irritant penetration enhancer (e.g., saponin, fusidate, azone, bile acid salts such as glycholate and cholate) and Apraclonidine HCl and is administered in accordance with the teachings of the present invention. The coadministration of Apraclonidine HCl and a highly irritant penetration enhancer in accordance with the teachings of of the present invention generally increases the bioavailability of the Apraclonidine HCl, allowing for administration of a reduced dose of API and more effective treatment of conditions for which treatment with Apraclonidine is useful, including short-term adjunctive therapy who require reduce intraocular pressure, glaucoma and ocular hypertension.

For example, a prior art course of treatment of high intraocular pessure is one drop (approximately 39 microliter) of Iopidine® per eye three times daily. In contrast, in embodiments of the present invention treatment of high intraocular pressure includes administering between about 1 microliter and about 39 microliters, preferably between 1 microliters and about 20 microliters of a composition including 0.5% Apraclonidine HCl and a highly irritant penetration enhancer to each eye three times daily. The composition is administered is a mist using a nebulizing device such as a device of the present invention.

### Dapiprazole

Dapiprazole (5,6,7,8-tetrahydro-3-[2-(4-o-toly-1-piperazinyl)ethyl]-s-triazolo [4,3-alpha] pyridine) is an alpha-adrenergic blocking agent having antimydriatic activity that is used to reduce the pupil size. Dapiprazole hydrochloride acts through blocking the alpha-adrenergic receptors in smooth muscle, producing miosis through an effect on the dilator muscle of the iris. Dapiprazole does not have any significant activity on ciliary muscle contraction and, therefore does not induce a significant change in the anterior chamber depth or the thickness of the lens. Dapiprazole is commercially available in an instillable solution including 0.5% (5 mg/ml) Dapiprazole under the tradename Rev-Eyes® of Bausch and Lomb (Rochester, N.Y., USA).

In an embodiment of the rpesent invention, a composition is provided comprising an ophthalmic carrier, a highly irritant penetration enhancer (e.g., saponin, fusidate, azone, bile acid salts such as glycholate and cholate) and Dapiprazole and is administered in accordance with the teachings of the present invention. The coadministration of Dapiprazole and a highly irritant penetration enhancer in accordance with the teachings of the present invention generally increases the bioavailability of the Dapiprazole, allowing for administration of a reduced dose of API and more effective treatment of conditions for which treatment with Dapiprazole is useful, including reversal of diagnostic mydriasis.

For example, a prior art course of treatment to reverse diagnostic mydriasis includes application of two drops (approximately 39 microliter each) of Rev-Eyes® followed after 5 minutes by an addional two drops to each eye. In contrast, in embodiments of the present invention reversal of diagnostic mydriasis includes administering between about 2 microliter and about 80 microliters, preferably between 2 microliters and about 40 microliters of a composition including 0.5% Dapiprazole and a highly irritant penetration enhancer to each eye a first time and after five minutes a second time. The composition is administered is a mist using a nebulizing device such as a device of the present invention.

### Dorzolamide

Dorzolamide (4S-trans)-4-(ethylamino)-5,6-dihydro-6-methyl-4H-thieno[2,3-b]thiopyran-2-sulfonamide 7,7-dioxide monohydrochloride) is a carbonic anhydrase inhibitor. Dorzolamide is commercially available in an instillable solution including 2% Dorzolamide under the tradename Trusopt® of Merck and Co., Inc. (Whitehouse Station, N.J., USA).

In an embodiment of the present invention, a composition is provided comprising an ophthalmic carrier, a highly irritant penetration enhancer (e.g., saponin, fusidate, qazone, bile acid salts such as glycholate and cholate) and Dorzolamide and is administered in accordance with the teachings of the present invention. The coadministration of Dorzolamide and a highly irritant penetration enhancer in accordance with the teachings of the present invention generally increases the bioavailability of the Dorzolamide, allowing for administration of a reduced dose of API and more effective treatment of conditions for which treatment with Dorzolamide is useful, including glaucoma and ocular hypertension.

For example, a prior art course of treatment to reduce ocular hypertension includes application of one drops (approximately 39 microliters) of Trusopt ® three times daily to each eye. In contrast, in embodiments of the present invention to reduce ocular hypertension includes administering between about 1 microliter and about 40 microliters, preferably between 1 microliters and about 20 microliters of a composition including 2% Dorzolamide and a highly irritant penetration enhancer to each eye three times daily. The composition is administered is a mist using a nebulizing device such as a device of the present invention.

### Timolol

Timolol ((−)-1-(tert-butylamino)-3-[(4-morpholino-1,2,5-thiadiazol-3-yl)oxyl]-2-propanol maleate) is a non-selective beta-adrenergic receptor blocking agent known to be exceptionally effective in reducing elevated as well as normal intraocular pressure, whether or not accompanied by glaucoma. Timolol Maleate is commercially available in an instillable solution including either 0.25% and 0.5% Timolol under the tradename Timoptic® of Merck and Co., Inc. (Whitehouse Station, N.J., USA).

In an embodiment of the present invention, a composition is provided comprising an ophthalmic carrier, a highly irritant penetration enhancer (e.g., saponin, fusidate, azone, bile acid salts such as glycholate and cholate) and Timolol and is administered in accordance with the teachings of the present invention. The coadministration of Timolol and a highly irritant penetration enhancer in accordance with the teachings of the present invention generally increases the bioavailability of the API, allowing for administration of a reduced dose and more effective treatment of conditions for which treatment with Timolol is useful, including ocular hypertension.

For example, a prior art course of treatment to reduce ocular hypertension includes application of one drop (approximately 39 microliters) of Timoptic® twice times daily to each eye. In contrast, in embodiments of the present invention to reduce oculawr hypertension includes administering between about 1 microliter and about 40 microliters, preferably between 1 microliters and about 20 microliters of a composition including 0.5% Timolol and a highly irritant penetration enhancer to each eye twice daily. The composition is administered is a mist using a nebulizing device such as a device of the present invention.

Dorzolamide with Timolol

Coadministration of Dorzolamide (4S-trans)-4-(ethylamino)-5,6-dihydro-6-methyl-4H-thieno[2,3-b]thiopyran-2-sulfonamide 7,7-dioxide monohydrochloride), a carbonic anhydrase inhibitor and timolol ((−)-1-(tert-butylamino)-3-[(4-morpholino-1,2,5-thiadiazol-3-yl)oxy]-2-propanol maleate) a beta-blocker is known to be exceptionally effective in treating open-angle glaucoma and ocular hypertension Dorzolamide HCl with Timolol Maleate is commercially available in an instillable solution including 2% Dorzolamide and 0.5% Timolol under the tradename Cosopt® of Merck and Co., Inc. (Whitehouse Station, N.J., USA).

In an embodiment of the present invention, a composition is provided comprising an ophthalmic carrier, a highly irritant penetration enhancer (e.g., saponin, fusidate, azone, bile acid salts seuch as glycholate and cholate), Dorzolamide and Timolol and is administered in accordance with the teachings of the present invention. The coadministration of Dorzolamide, Timolol and a highly irritant penetration enhancer in accordance with the teachings of the present invention generally increases the bioavailability of the APIs, allowing for administration of a reduced dose and more effective tr3eatment of conditions for which treatment with Dorzolamide and Timolol is useful, including glaucoma and ocular hypertension.

For example, a prior art course of treatment to reduce ocular hypertension includes application of one drops (approximately 39 microliters) of Cosopt ® twice times daily to each eye. In contrast, in embodiments of the present invention to reduce ocular hypertension includes administering between about 1 microliter and about 40 microliters, preferably between 1 microliters and about 20 microliters of a composition including 2% Dorzolamide, 0.5% Timolol and a highly irritant penetration enhancer to each eye twice daily. The composition is administered is a mist using a nebulizing device such as a device of the present invention.

### Unoprostone Isopropyl

Unoprostone Isopropyl (isopropyl (+)-(Z)-7-[(1R, 2R, 3R, 5S)-3,5-dihydroxy-2-(3-oxodecyl) cyclopentyl]-5-heptenoate) is the isopropyl ester of unoprostone, a PGF2alpha analog with a 13,14-dihydro-15-keto modification and a two-carbon extension of the aliphatic lower side chain approved for teatment of open-angle extension of the aliphatic lower side chain approved for treatment of open-angle glaucoma or ocular hypertension. Unoprostone Isopropyl is commercially available in an instillable solution including 0.15% Unoprostone Isopropyl under the tradename Rescula® of CIBA Vision, A Novartis Company (Duluth, Ga., USA).

In an embodiment of the present invention, a composition is provided comprising an ophthalmic carrier, a highly irritant penetration enhancer (e.g., saponin, fusidate, azone, bile acid salts such as glycholate and cholate) and Unoprostone Isopropyl and is administered in accordance with the teachings of the present invention. The coadministration of Unoprostone Isopropyl and a highly irritant penetration enhancer in accordance with the teachings of the present invention generally increases the bioavailability of the Unoprostone Isopropyl, allowing for administration of a reduced dose of API and more effective treatment of conditions for which treatment with Unoprostone Isopropyl is useful, including glaucoma and ocular hypertension.

For example, a prior art course of treatment to reduce ocular hypertension includes application of one drops (approximately 39 microliters) of Rescula® twice daily to each eye. In contrast, in embodiments of the present invention to reduce ocular hypertension includes administering between about 1 microliter and about 40 microliters, preferably between 1 microliters and about 20 microliters of a composition including 0.15% Unoprostone Isopropyl and a highly irritant penetration enhancer to each eye twice daily. the composition is administered is a mist using a nebulizing device such as a device of the present invention.

### Levobunolol

Levobunolol ((−)-5-[3-(tert-Butylamino)-2-hydroxypropoxy]-3, 4-dihydro-1(2H)-naphthalenone hydrochloride) is a noncardioselective beta-adrenoceptor blocking agent, equipotent at both beta1 and beta2 receptors. Levobunolol is greater than 60 times more potent than its dextro isomer in its beta-blocking activity, yet equipotent in its potential for direct myocaridal depression. Levobunolol HCl does not have significant local anesthetic (membrane-stabilizing) or intrinsic sympathomimetic activity. Levobunolol HCl is approved for treatment of lowering intraocular pressure and may be used in patients with chronic open-angle glaucoma or ocular hypertension. Levobunolol is commercially available in an instillable solution including 0.25% and 0.5% Levobunolol under the tradename Betagan Liquiflim® of Allergan Inc (Irvine, Calif., USA).

In an embodiment of the present invention, a composition is provided comprising an ophthalmic carrier, a highly irritant penetration enhancer (e.g., saponin, fusidate, azone, bile acid salts such as glycholate and cholate) and Levobunolol and is administered in accordance with the teachings of the present invention. the coadministration of Levobunolol and a highly irritant penetration enhancer in accordance with the teachings of the present invention generally increases the bioavailability of the Levobunolol, allowing for administration of a reduced dose of API and more effective treatment of conditions for which treatment with Levobunolol is useful, including glaucoma and ocular hypertension.

For example, a prior art course of treatment to reduce ocular hypertension includes application of one drops (approximately 39 microliters) of Betagan Liquifilm® twice daily to each eye. In contrast, in embodiments of the present invention to reduce ocular hypertension includes administering between about 1 microliter and about 40 microliters, preferably between 1 microliters and about 20 microliters of a composition including 0.25% Levobunolol and a highly irritant penetration enhancer to each eye twice daily. The composition is administered is a mist using a nebulizing device such as a device of the present invention.

### Betaxolol

Betaxolol ((±)-1-[p-[2-(cyclopropylmethoxy)ethyl]phenoxy]-3-(isopropylamino)-2-propanol hydrochloride) is a cardioselective (beta-1-adrenergic) receptor blocking agent, does not have significan membrane-stabilizing (local anesthetic) activity and is devoid of intrinsic sympathomimetic action. Betaxolol

Is approved for reducing elevated intraocular pressure, whether or not accompanied by glaucoma. Levobunolol is commercially available in an instillable suspension including 0.25% Betaxolol under the tradename Betoptic S® of alcon USA (Fort Worth, Tex., USA).

In an embodiment of the present invention, a composition is provided comprising an ophthalmic carrier, a highly irritant penetration enhancer (e.g., saponin, flusidate, azone, bile acid salts such as glycholate and cholate) and Betaxolol and is administered in accordance with the teachings of the present invention. the coadministration of Betaxolol and a highly irritant penetration enhancer in accordance with the teachings of the present invention generally increases the bioavailability of the Betaxolol, allowing for administration of a reduced dose of API and more effective treatment of conditions for which treatment with Betaxolol is useful, including glaucoma and ocular hypertension.

For example, a prior art course of treatment to reduce ocular hypertension includes application of one drops (approximately 39 microliters) of Betaxolol® twice daily to each eye. In contrast, in embodiments of the presnet invention to reduce ocular hypertension includes administering between about 1 microliter and about 40 microliters, preferably between 1 microliters and about 20 microliters of a composition including 0.25% Betaxolol and a highly irritant penetration enhancer to each eye twice daily. the composition is administered is a mist using a nebulizing device such as a device of the present invention.

### Pilocarpine

Pilocarpine (3S, 4R)-3-Ethyldihydro-4-[(1-methyl-1H-imidazole-5-yl) methyl]-2(3-H)-furanone) is a direct acting cholinergic (parasympathomimetic) agent causing pupillary constriction. By mimicking acetylcholine, pilocarpine acts as a stimulant of the parasympathetic nervous system promoting the increases the outflow of fluid from the eye with a concomitant reduction of intraocular pressure. Pilocarpine is approved for the treatment of primary open-angle glaucoma and also to lower intraocular pressure prior to surgery for acute angle-closure glaucoma. Pilocarpine is commercially available in an instillable suspension including 1%, 2% or 4% mg/mL. Pilocarpine under the tradename Pilagan Liquifilm® of Allergan Inc (Irvine, Calif., USA).

In an embodiment of the present invention, a composition is provided comprising an ophthalmic carrier, a highly irritant penetration enhancer (e.g., saponin, fusidate, azone, bile acid salts such as glycholate and cholate) and Pilocarpine and is administered in accordance with the teachings of the present invention. The coadministration of Pilocarpine and a highly irritant penetration enhancer in accordance with the teachings of the present invention generally increases the bioavailability of the Pilocarpine, allowing for administration of a reduced dose of API and more efective treatment of conditions for which treatment with Pilocarpine is useful, including glaucoma, ocular hypertension and mydriasis.

For example, a prior art course of treatment to reduce ocular hypertension includes application of two drops (approximately 39 microliters each) of Pilagan Liquifilm® 1% up to four times daily to each eye. In contrast, in embodiments of the present invention to reduce ocular hypertension includes administering between about 1 microliter and about 80 microliters, preferably between 1 microliters and about 40 microliters of a composition including 1% Pilocarpine and a highly irritant penetration enhancer to each eye twice daily. the composition is administered is a mist using a nebulizing device such as a device of the present invention.

### Echothiphate Iodide

Echothiphate Iodide ((2-mercaptoethyl) trimethylammonium iodide O,O-diethyl phosphorothioate) is a long-acting cholinesterase inhibitor which enhances the effect of endogenously liberated acetylcholine in iris, ciliary muscle, and other parasympathetically innervated structures of the eye when applied topically, thereby causing miosis, increase in facility of outflow of aqueous humor, fall in intraocular pressure, and potentiation of accommodation. Echothiophate iodide will depress both plasma and erythrocyte cholinesterase levels in most patients after a few weeks of therapy. Echothiophate iodide is approved for the treatment of chronic open-angle glaucoma, subacute or chronic angle-closure glaucoma after iridectomy or where surgery is refused or contraindicated and certain non-uveitic secondary types of glaucoma, especially glaucoma following cataracet surgery. Kits for preparation of Echothiphate Iodide instillable solutions are commercially available under the tradename Phospholine Iodide® of Wyeth Pharmaceuticals (Collegeville, Pa., USA)

In an embodiment of the present invention, a composition is provided comprising an ophthalmic carrier, a highly irritant penetration enhancer (e.g., saponin, fusidate, azone, bile acid salts such as glycholate and cholate) and Echothiophate iodide and is administered in accordance with the teachings of the present invention. The coadministration of Echothiophate iodide and a highly irritant penetration enhancer in accordance with the teachings of the present invention generally increases the bioavailability of the Echothiophate iodide, allowing for administration of a reduced dose of API and more effective treatment of conditions for which treatment with Echothiophate iodide is useful, including glaucoma and ocular hypertension.

For example, a prior art course of treament to reduce ocular hypertension includes application of one drop (approximately 39 microliters) of Phospholine Iodide® 0.125% twice daily to each eye. In contrast, in embodiments of the present invention to reduce ocular hypertension includes administering between about 1 microliter and about 40 microliters, preferably between 1 microliters and about 20 microliters of a composition including 0.125% Phospholine Iodide and a highly irritant penetration enhancer to each eye twice daily. The composition is administered is a mist using a nebulizing device such as a device of the present invention.

### Latanoprost

Latanoprost (isopropyl-(Z)-7[(1R, 2R, 3R, 5S)3, 5-dihydroxy-2-[(3R)-3-hydroxy-5-phenylpentyl]cyclopentyl]-5-heptenoate) is a prostanoid selective FP receptor agonist that is believed to reduce the intraocular presssure (IOP) by increasing the outflow of aqueous humor. Studies in animals and man suggest that the main mechanism of action is increased uveoscleral outflow. Latanoprost is approved for the treatment of elevated intraocular pressure in patients with open-angle glaucoma or ocular hypertension. Latanoprost is commercially available in 0.005% instillable solutions under the tradename Xalatan® of Pfizer (New York, N.Y., USA).

In an embodiment of the present invention, a composition is provided comprising an ophthalmic carrier, a highly irritant penetration enhancer (e.g., saponin) fusidate, azone, bile acid salts such as glycholate and cholate) and Latanoprost and is administered in accordance with the teachings of the present invention. The coadministration of Latanoprost and a highly irritant penetration enhancer in accordance with the teachings of the present invention generally increases the bioavailability of the Latanoprost, allowing for administration of a reduced dose of API and more effective treatment of conditions for which treatment with Latanoprost is useful, including glaucoma and ocular hypertension.

For example, a prior art course of treatment to reduce ocular hypertension includes application of one drop (approximately 39 microliters) of Xalatan® once a day to each eye. In contrast, in embodiments of the present invention to reduce ocular hypertension includes administering between about 1 microliter and about 40 microliters, preferably between 1 microliters and 20 microliters of a composition including Latanoprost and a highly irritant penetration enhancer to each eye once a day. The composition is administered is a mist using a nebulizing device such as a device of the present invention.

### Bimatoprost

Bimatoprost ((Z)-7-[(1 R, 2 R, 3 R, 5 S)-3, 5-dihydroxy-2-[1 E, 3 S)-3-hydroxy-5-phenyl-1-pentenyl]cyclopentyl]-5-N-ethylheptenamide) is a prostamide, a synthetic structural analog of prostaglandin with ocular hypotensive activity. It selectively mimics the effects of naturally occurring substances, prostamide. Bimatoprost is believed to lower intraocular pressure in humans by increasing outflow of aqueous humor through both the trabecular meshwork and uveoscleral routes. Bimatoprost is approved for the treatment of elevated intraocular pressure in patients with open angle glaucoma or ocular hypertension. Bimatoprost is commercially available in 0.03% instillable solutions under the tradename Lumigan® of of Allergan Inc (Irvine, Calif., USA).

In an embodiment of the present invention, a composition is provided comprising an ophthalmic carrier, a highly irritant penetration enhancer (e.g., saponin, fusidate, azone, bile acid salts such as glycholate and cholate) and Bimatoprost and is administered in accordance with the teachings of the present invention. The coadministration of Bimatoprost and a highly irritant penetration enhancer in accordance with the teachings of the present invention generally increases the bioavailability of the Bimatoprost, allowing for administration of a reduced dose of API and more effective treatment of conditions for which treatment with Bimatoprost is useful, including glaucoma and ocular hypertension.

For example, a prior art course of treatment to reduce ocular hypertension includes application of one drop (approximately 39 microliters) of Lumigan® once a day to each eye. In contrast, in embodiments of the present invention to reduce ocular hypertension includes administering between about 1 microliter and about 40 microliters, preferably between 1 microliters and about 20 microliters of a composition including Bimatoprost and a highly irritant penetration enhancer to each eye once a day. The composition is administered is a mist using a nebulizing device such as a device of the present invention.

### Flurbiprofen Sodium

Flurbiprofen Sodium (Sodium (±)-2-(2-fluoro-4-biphenyl)-propionate dihydrate) is a phenylalkanoic acids having analgensic, antipyretic, and anti-inflammatory activity in animal inflammatory diseases. Its mechanism of action is believed to be through inhibition of the cyclo-oxygenase enzyme that is essential in the biosynthesis of prostaglandins. Prostaglandins have been shown in many animal models to be mediators of certain kinds of intraocular inflammation. In studies performed on animal eyes, prostaglandins have been shown to produce disruption of the blood-aqueous humor barrier, vasodilation, increased vascular permeability, leukocytosis, and increased intraocular pressure. Prostaglandins also appear to play a role in the miotic response produced during ocular surgery by constricting the iris sphincter independently of cholinergic mechanisms. Flurbiprofen Sodium is approved for the inhibition of intraoperative miosis Flurbiprofen Sodium is commercially available in 0.03% instillable solutions under the tradename Ocufen® of Allergan Inc (Irvine, Calif., USA).

In an embodiment of the present invention, a composition is provided comprising an ophthalmic carrier, a highly irritant penetration enhancer (e.g., saponin, fusidate, azone, bile acid salts such a glycholate and cholate) and Flurbiprofen Sodium and is administered in accordance with the teachings of the present invention. The coadministration of Flurbiprofen Sodium and a highly irritant penetration enhancer in accordance with the teachings of the present invention generally increases the bioavailability of the Flurbiprofen Sodium, allowing for administration of a reduced dose of API and more effective treatment of conditions for which treatment with Flurbiprofen Sodium is useful, including inhibition of intraoperative miosis.

For example, a prior art course of treatment to inhibit intraoperative miosis includes application of one drop (approximately 39 microliters) of Ocufen® once every half hour four times starting two hours before surgery to an appropriate eye. In contrast, in embodiments of the present invention to inhibit intraoperative miosis includes administering between about 1 microliter and about 40 microliters, preferably between 1 microliters and about 20 microliters of a composition including Flurbiprofen Sodium and a highly irritant penetration enhancer once every half hour four times starting two hours before surgery to an appropriate eye. The composition is administered is a mist using a nebulizing device such as a device of the present invention.

### Prednisolone Acetate

Prednisolone Acetate (1,4-pregnadiene-11-beta, 17-alpha-21-triol-3,20-dione 21-acetate). Corticosteroids inhibit the inflammatory response to a variety of inciting agents and probably delay or slow healing. Corticosteroids inhibit the edema, fibrin deposition, capillary dilation, leukocyte migration, capillary proliferation, fibroblast proliferation, deposition of collagen, and scar formation associated with inflammation. There is no generally accepted explanation for the mechanism of action of ocular corticosteroids. However, corticosteroids are thought to act by the induction of phospholipase A2 inhibitory proteins, collectively called lipocortins. It is postulated that these proteins control the biosynthesis of potent mediators of inflammation such as prostaglandins and leukotrienes by inhibiting the release of their common precursor arachidonic acid. Arachidonic acid is released from membrane phospholipids by phospholipase A2. Prednisolone Acetate is approved for steroid responsive inflammatory conditions of the palpebral and bulbar conjunctiva, cornea, and anterior segment of the globe such as allergic conjunctivitis, acne rosacea, superficial punctate keratitis, herpes zoster keratitis, iritis, cyclitis, selected infective conjunctivitides, when the inherent hazard of steroid use is accepted to obtain an advisable diminution in edema and inflammation; corneal injury from chemical radiation, or thermal burns, or penetration of foreign bodies. Prednisolone Acetate is commercially available in a 1% instillable suspension under the tradename Pred Forte® of Allergan Inc (Irvine, Calif., USA).

In an embodiment of the present invention, a composition is provided comprising an ophthalmic carrier, a high irritant penetration enhancer (e.g., saponin, fusidate, azone, bile acid salts such as glycholate and cholate) and Prednisolone Acetate and is administered in accordance with the teachings of the present invention. The coadministration of Prednisolone Acetate and a highly irritant penetration enhancer in accordance with the teachings of the present invention generally increases the bioavailability of the Prednisolone Acetate, allowing for administration of a reduced dose of API and more effective treatment of conditions for which treatment with Prednisolone Acetate is useful, including inhibition of inflammation.

For example, a prior art course of treatment of ocular inflammation includes application of one or two drop (approximately 39 microliters each) of Pred Forte® once every hour during the day and once every two hours in during the night to an appropriate eye. In contrast, in embodiments of the present invention to treat ocular inflammation includes administering between about 1 microliter and about 40 microliters, preferably between 1 microliters and about 20 microliter and about 40 microliters, preferably between 1 microliters and about 20 microliters of a composition including Prednisolone Acetate and a highly irritant penetration enhancer once every hour during the day and once every two hours during the night to an appropriate eye. The composition is administered is a mist using a nebulizing device such as a device of the present invention.

### Dexamethasone

Dexamethasone (9-fluoro-11b, 17, 21-trihydroxy-16a-methylpregna-1, 4-diene-3,20-dione). Dexamethasone is a synthetic glucocorticod analog. Glucocorticoids, naturally occurring and synthetic, are adrenocortical steroids that cause varied metabolic effects and modify the body's immune responses to diverse stimuli. Naturally occurring glucocorticoids (hydrocortisone and cortisone), which also have sodium-retaining properties, are used as replacement therapy in adrenocortical deficiency states. Synthetic analogs including dexamethasone are primarily used for their anti-inflammatory effects in disorders of many organ systems. At equipotent anti-inflammatory doses dexamethasone almost completely lacks the sodium-retaining property of hydrocortisone and closely related derivatives of hydrocortisone. Dexamethasone is approved for Allergic states such as control of severe or incapacitating allergic conditions intractable to adequate trials of conventional treatment in asthma, atopic dermatitis, contact dermatitis, drug hypersensitivity reactions, perennial or seasonal allergic rhinitis, and serum sickness; dermatologic diseases such as Bullous dermatitis herpetiformis, exfoliative erythroderma, mycosis fungoides, pemphigus, and severe erythema multiforme (Stevens-Johnson syndrome); endocrine disorders such as primary or secondary adrenocortical insufficiency (hydrocortisone or cortisone is the drug of choice; may be used in conjunction with synthetic mineralocorticoid analogs where applicable; in infancy mineralocorticoid supplementation is of particular importance), congenital adrenal hyperplasia, hypercalcemia associate with cancer, and nonsuppurative thyroiditis; gastrointestinal diseases to tide the patient over a critical period of the disease in regional enteritis and ulcerative colitis; hematologic disorders such as acquired (autoimmune) hemolytic anemia, congenital (erythroid) hypoplastic anemia (Diamond-Blackfan anemia), idiopathic thrombocytopenic purpura in adults, pure red cell aplasia, and selected cases of secondary thrombocytopenia. Miscellaneous: Diagnostic testing of adrenocortical hyperfunction, trichinosis with neurologic or myocardial involvement, tuberculous meningitis with subarachnoid block or impending block when used with appropriate antituberculous chemotherapy; neoplastic diseases for the palliative management of leukemias and lymphomas; for renal diseases to induce a diuresis or remission of proteinuria in diopathic nephrotic syndrome or that due to lupus erythematosus; respiratory diseases such Berylliosis, fulminating or disseminated pulmonary tuberculosis when used concurrently with appropriate antituberculous chemotheapy, idiopathic cosinophilic pneumonias, symptomatic sarcoidosis; rheumatic disorders as adjunctive therapy for short-term administation (to tide the patient over an acute episode or exacerbation) in acute gouty arthritis, acute rheumatic carditis, ankylosing spondylitis, psoriatic arthritis, rheumatoid arthritis, including juvenile rheumatoid arthritis (selected cases may require low-dose maintenance therapy) and for the treatment of dermatomyositis, polymyositis, and systemic lupus erythematosus; in the nervous system to treat acute exacerbations of multiple sclerosis, cerebral edema associated with primary or metastatic brain tumor, craniotomy, or head injury and ophthalmic diseases such as cyclitis, Herpes zoster ophthalmicus, iridocyclitis, iritis, sympathetic ophthalmia, temporal arteritis, uveitis, nonpurulent conjunctivitis (including vernal, allergic, catarrhal, especially where allergy inflammatory reactions, recurrent marginal ulceration of toxic or allergic etiology, thermal burns, chemical burns and ocular inflammatory conditions unresponsive to topical corticosteroids. Dexamethasone is commercially available in a 0.1% instillable suspension under the tradename Maxidex™ of Alcon Inc (Fort Worth, Tex., USA).

In an embodiment of the present invention, a composition is provided comprising an ophthalmic carrier, a highly irritant penetration enhancer (e.g., saponin, fusidate, azone, bile acid salts such as glycholate and cholate) and Dexamethasone and is administered in accordance with the teachings of the present invention. The coadministration of Dexamethasone and a highly irritant penetration enhancer in accordance with the teachings of the present invention generally increases the bioavailability of the Dexamethasone, allowing for administration of a reduced dose of API and more effective treatment of conditions for which treatment with Dexamethasone is useful, including ocular inflammatory conditions.

For example, a prior art course of treatment of ocular inflammation includes application of one or two drop (approximately 39 microliters each) of Maxidex™ once 30-60 minutes to an appropriate eye. In contrast, in embodiments of the present invention to treat ocular inflammation includes administering between about 1 microliter and about 40 microliters, preferably between 1 microliters and about 20 microliters of a composition including Dexamethasone and a highly irritant penetration enhancer once every 30-60 minutes to an appropriate eye. The composition is administered is a mist using a nebulizing device such as a device of the present invention.

### Triamcinolone Acetonide

Triamcinolone acetonide (9-Fluoro-11b, 16a, 17, 21-tetrahydroxypregna-1, 4-diene-3, 20-dione cyclic 16, 17-acetal). Triamcinolone acetonide is a potent steroid. Triamcinolone acetonide is approved as an injection into the vitreous cavity for treating ocular inflammation and swelling, lile cystoid macular edema, diabetic macular edema and wet AMD. Triamcinolone acetonide is commercially available as an intravitreal injectable solution from Bristol-Myers Squibb Company (New York, N.Y., USA).

In an embodiment of the present invention, a composition is provided comprising an ophthalmic carrier, a highly irritant penetration enhancer (e.g., saponin, fusidate, azone, bile acid salts such as glycholate and cholate) and Triamcinolone acetonide and is administered in accordance with the teachings of the present invention. The coadministration of Triamcinolone acetonide and a high irritant penetration enhancer in accordance with the teachings of the present invention allows topical application of Triamcinolone acetonide instead of the unpleasant intravitreal injection known in the art, allowing effective treatment of conditions for which treatment with Triamcinolone acetonide is useful, including ocular inflammatory conditions.

For example, a prior art course of treatment of ocular inflammation includes injection of 4 mg Triamcinolone acetonide into the eye. In contrast, in embodiments of the present invention to treat ocular inflammation includes administering between 4 mg and 20 mg Triamcinolone acetonide in between about 1 microliter and about 100 microliters, preferably between 1 microliters and about 50 microliters of a composition including Triamcinolone acetonide and a highly irritant penetrartion enhancer to an appropriate eye. The composition is administered is a mist using a nebulizing device such as a device of the present invention.

### Nepafenac

Nepafenac, the amide analog of 2-amino-3-benzoylbenzeneacetic acid (amfenac), has been extensively studied (Takahashi et al. Invest. Ophthalmol. Vis. Sci. 2003, 44(1), 409-415; Gamache et al. Inflammation 2000, 24(4), 357-370; de Souza et al. Eye 2005, Jun. 17; Kapin et al. Inflammation 2003, 27(5) 281-291; Ke et al. Inflammation 2000, 24(4), 371-384; Koevary Curr. Drug Metabl. 2003, 4(3) 213-222).

Nepafenac is characterized in having excellent permeability into the eye through the cornea when applied topically. When inside the eye, nepafenac is metabolized into amfenac, a high potent COX-1 and COX-2 inhibiting non-steroid anti-inflammatory drug. Compositions including nepafenac are considered to be useful for the treatment ocular inflammation, postoperative ocular pain, posterior segment edema, retinoblastoma, retinal edema, prostaglanding formation and for COX-1 and COX-2 inhibition.

In an embodiment of the present invention, a composition is provided comprising an ophthalmic carrier, a highly irritant penetration enhancer (e.g., saponin, fusidate, azone, bile acid salts such as glycholate and cholate) and nepafenac and is administered in accordance with the teachings of the present invention. The coadministration of nepafenac and a highly irritant penetration enhancer generally allows for increased bioavailability and more effective treatment of conditions for which treatment with Nepafenac is useful including ocular inflammation, postoperative ocular pain, posterior segment edema, retinoblastoma, retinal edema, glaucoma, prostaglandin formation, retinal and choroidal neovascularization and for COX-1 and COX-2 inhibition.

Embodiments of a composition of the present invention include an API and/or a penentration enhancer in an ophthalmically acceptable carrier and optionally other ingredients.

Ophthalmically acceptable carriers are generally sterile, essentially free of foreign particles, and have a pH in the range of 5-8. Preferably, the pH is as close to the pH of tear fluid (7.4) as possible. Ophthalmically acceptable carriers are, for example, sterile isotonic solutions such as isotonic sodium chloride or boric acid solutions. Such carriers are typically aqueous solutions contain sodium chloride or boric acid. Also useful are phosphate buffered saline (PBS) solutions.

In embodiments of the present invention, a composition includes an effective amount of an active pharmaceutical incredient (API). In embodiments of the present invention the API is a peptide or protein. An effective amount of an API, as used herein, means an amount needed to achieve the desired outcome prophylactic, therapeutic, pharmaceutical or cosmeceutical effect, which is generally to prevent, alleviate or ameliorate the condition or symptoms of the condition which is being treated. Determination of the effective amount, and consequently the dose and dose frequency, is within the capability of one skilled in the art, especially in light of the detailed disclosure provided herein. Factors in determining the effective amount vary with severity of the condition as well as such factors as the concentration of the active pharmaceutical ingredient or ingredients, the subject being treated, the severity of the condition, the age, body weight and response of an individual patient and the judgment of the prescribing physician. Generally, the concentration of an API does not exceed 10% (w/v, e.g., mg μl⁻¹) and is generally not more than about 5%, not more than about 2.5% and even not more than about 1%.

In embodiments of the present an API is encapsulated in or contained within some structure, whether to protect the API or to provide another desired property such as increased penetration or adhesion to a surface. Such structures include beads, ethosome (Novel Therapeutic Technologies, Zichron Yakov, Israel), liposomes, lipospheres, micelles, microcapsule, microspheres, nanocapsules, nanoparticles, nanospheres. A review of suitable such structures is found, for example, in Kumar, M. N. V. R. J. Pharm. Pharmaceut. Sci. 2000, 3(2), 234-258.

It is often desired to provide a composition with additional useful properties. Therefore, in some embodiments, a composition of the present invention includes, in addition to a penetration enhancer and/or a protein or peptide active ingredient in a ophthalmically acceptable carrier, at least one additional component. It is important to note that in some cases a specific additional component also serves as a component of the carrier or serves two or more additional functions. Typical additional components include but are not limited to bioadhesives, buffering agents, chelating agents, humectants, pH-adjusting agents, preservatives, solubilizers, viscosity modifiers and vitamins.

In embodiments of the present invention, a composition includes a bioadhesive, especially a bioadhesive polymer, especially a bioadhesive that is useful to keep and administered API a longer than usual time on rhe cornea. Suitable bioadhesives include but are not limited to polyvinyl alcohol, thiolated poly acrylic acid, carbomer and gellan gum.

In embodiments of the present invention, a composition includes a buffering agent. Suitable buffering agents include but are not limited to borate buffers, citrate buffers, acetic acid/sodium acetate buffers and a phosphoric acid/sodium phosphate buffers.

In embodiments of the present invention, a composition includes a humectant. Suitable humectants include but are not limited to ammonium lactate, guanidine, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine and accetamide monoethanolamine, urea, or a combination thereof.

In embodiments of the present invention, a composition includes a pH-adjusting agent. Suitable pH-adjusting agents include but are not limited to adipic acid, borics acid, citric acid, glycine, calcium hydroxide, magnesium aluminometasilicates, hydrochloric acid, lactic acid, phosphoric acid, sodium hydroxide, sorbic acid, sulfuric acid and tartaric acid, derivatives thereofs, salts thereofs or combinations thereof.

In embodiments of the present invention, a composition include preservative. Suitable preservatives include but are not limited to alkanols, C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorobutanol, chlorocresol, citric acid, cocoa butter, coconut oil, diazolidinyl urea, diisopropyl adipate, dimethyl polysiloxane, DMDM hydantoin, disodium EDTA (ethylenediamine tetraacetate), EDTA salts, EDTA fatty acid conjugates, ethanol fatty acids, fatty alcohols, hexadecyl alcohol, hydroxybenzoate esters, iodopropynyl butylcarbamate, isononyl iso-nonanoate, isothiazolinone, jojoba oil, lanolin oil, methylparaben, mineral oil, oleic acid, olive oil, parabens, polyethers, polyoxypropylene butyl ether, polyoxypropylene cetyl ether, potassium sorbate, propylene glycols, propylparaben, silicone oils, sodium propionate, sodium benzoate, sodium bisulfite, sorbic acid, sorbates, stearic fatty acid, vitamin E, vitamin F, acetate and derivativ es, esters, salts and mixtures thereof.

In embodiments of the present invention, a composition include solubilizer. suitable solubilizers include but are not limited to citric acid, ethylenediamine-tetraacetate, sodium meta-phosphate, succinic acid, urea, cyclodextrin, polyvinylpyrrolidone, diethylammonium-ortho-benzoate, micelle-forming solubilizers, TWEENS, SPANS, polyoxyethylene sorbitan fatty acid ester, polyoxyethylene n-alkyl ethers, n-alkyl amine n-oxides, poloxamers, phospholipids and cyclodextrines.

In embodiments of the present invention, a composition includes a viscosity modifier. A suitable viscosity modifier is methylcellulose.

In embodiments of the present invention, a composition includes a vitamin. Suitable vitamins include but are not limited to retinoids, vitamin A, retinol, retinal, retinyl palmitate, retinoic acid, tretinoin, iso-tretinoin, vitamin E, tocopherol, vitamin C, L-ascorbic acid, vitamin B₃, niacinamide, alpha hydroxy acids, glycoli acid, lactic acid, tartaric acid, malic acid, citric acid, beta hydroxy acids, salicylic acid, esters thereof and derivatives thereof.

In embodiments of the present invention, a composition is formulated to ophthalmically deliver an active pharmaceutical ingredient. It is therefore preferred that such composition be packages in a packaging material and identified in print, in or on the packaging material, as an ophthalmically deliverable composition for use for a need. By “need” is meant a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. A specific condition and specific use is dependent on the exact formulation of a specific, including the identity and amount of the one or more active pharmaceutical ingredients therein.

Aspects of the present invention are implemented using any nebulizing device known in the art for ophthalmic administration of a pharmaceutical composition such as described in the introduction herein. Most preferably such aspects are implemented using an embodiment of a device of the present invention.

Embodiments of a device of the present invention include many features useful for an ophthalmic delivery system including accepting off-the-shelf bottles, (and also bottles and unidose cartridges), dosage control, production of nano-size droplets to overcome protective mechanisms of the eye by not activating the blinking reflex and not stimulating tear generation sensory receptors, employ a computerized system to program a predetermined medication-application regimen, call or beep the user, at the schedule time for medication application, store data related to the medication application, and communicate with a computer or a health clinic concerning the treatment regimen for follow-up and evaluation. Embodiments of a device of the present invention include a feature to direct a generated mist only at an open eye, increasing dosage accuracy and reducing composition wastage.

Embodiments of a device of the present invention include a self-sterlizing feature increasing the safey of the device and allowing the device to be easily used in hospitals and in situation that require high-throughput administration of a pharmaceutical composition.

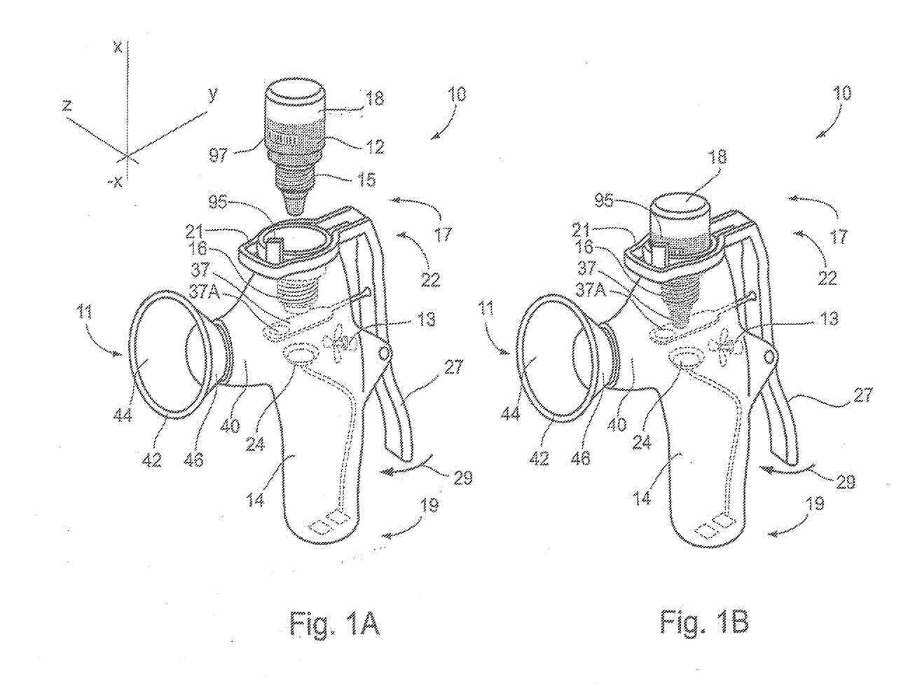

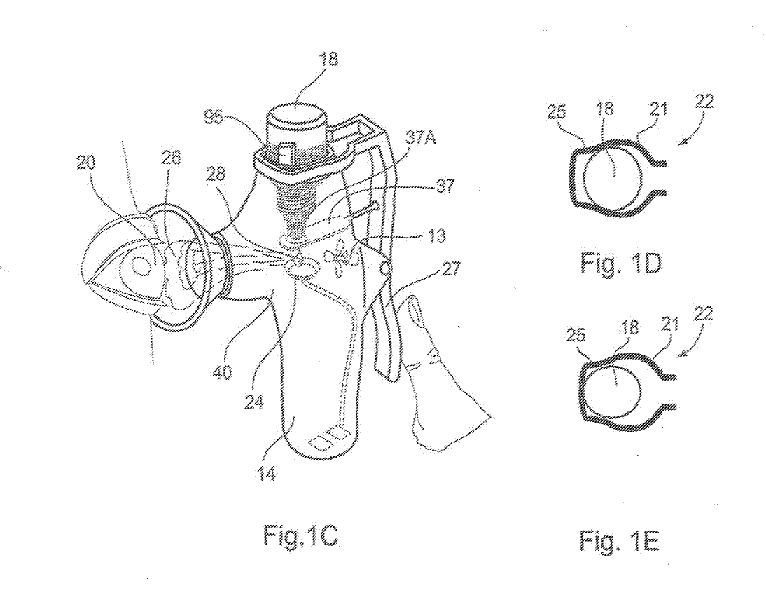

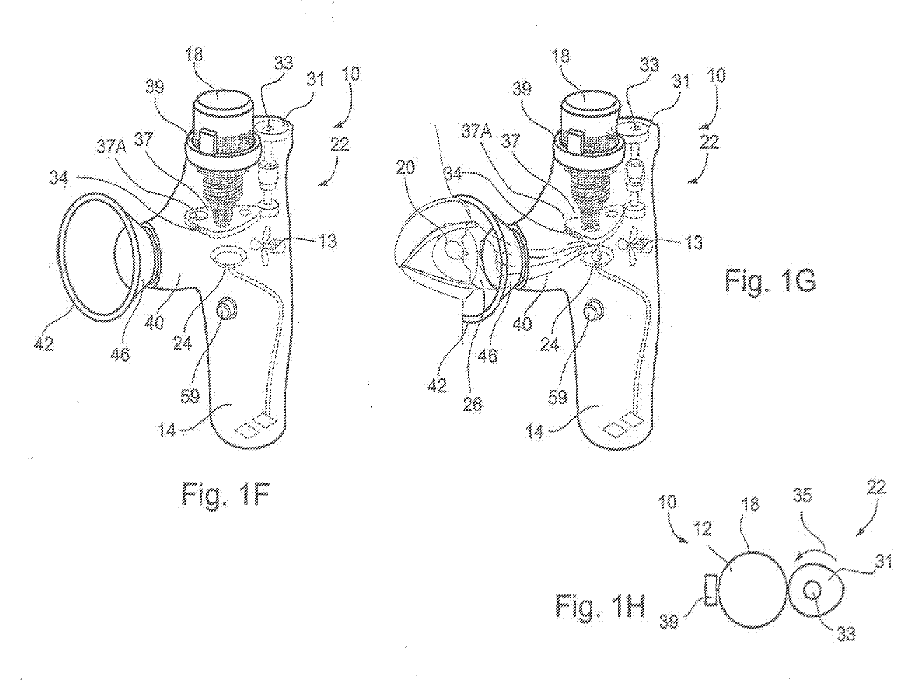

Referring now to the drawings, FIGS. 1A-1H schematically depict an embodiment of a device 10 for applying a pharmaceutical composition 12 to an eye 20, in accordance with the present invention. As ciscussed above a preferred pharmaceutical composition 12 is an embodiment of a pharmaceutical composition of the prewent invention such as a pharamaceutical composition including a peptide or protein API, a pharmaceutical composition including a peptide or protein API for treatment of nerves and especially of the central nervous system or a pharmaceutical composition including irritant penetration enhancers, whether inherently or by virtue of a high concentration. In FIG. 1, pharmaceutical composition 12 is a liquid, but embodiments of the present invention are configured to administer by nebulization pharmaceutical compositions in other states such as solids, semi-solids, gels and the like.

As seen in FIGS. 1A-1E, device 10 is formed of a body 14, which defines an x;y;z coordinate system, with—x being the direction of gravity, upper and lower ends 17 and 19 respectively, along the x-axis, with respect to the direction of gravity, and a proximal end 11, along the y-axis, with respect to eye 20 (FIG. 1C). Body 14 includes a mist-generator head 40, which includes:
a holding sturcture 21, generally at upper end 17, for receiving an off-the-shelf container 18 in an inverted position, wherein container 18 contains pharmaceutical composition 12;
an adaptor 16, for providing fluid communication with off-the-shelf container 18;
an actuating mechanism 22, adapted to communicate in mechanical communication with off-the-shelf container 18, for causing a predetermined amount of pharmaceutical composition 12 to be issed therefrom; and
a nebulizer 24, in cluif communication with off-the-shelf container 18, for receiving and nebulizing a pharmaceutical composition 12, to form a mist 26 (FIG. 1C).

Container 18 is inverted and fitting into adapter 16, which may be an inner thread, adapted to fit onto an external thread 15, of container 18 (FIG. 1A).

Alternatively, adapter 16 is a rubber-like opening, for forming a tight seal around container 18.

Actuating mechanism 22 may be a manual device, for example, operated by a lever 27. The issue of pharmaceutical composition 12 takes place by pressing lever 27 in the direction indicated by arrow 29 (FIGS. 1A and 1B).

As seen in FIGS. 1D and 1E, container 18 is formed of a flexible material and holding strucrture 21 includes a narrow portion 25 so that the pressing of lever 27 causes container 18 to be squeezed into narrow portion 25. Alternatively, container 18 is formed of a rigid material, but contains a small air vent, so that narrow portion 25 need not be used; pharmaceutical composition 12 flows out in the direction of gravity whener container 18 is inverted.

Additionally, as seen in FIG. 1A, a plate 37 with a hole 37A, is preferably controlled by actuating mechanism 22 and includes “on” and “off” positions. In the “off” position of FIG. 1A plate 37 presses against container 18 so as to keep pharmaceutical composition 12 from issuing.

As seen in FIG. 1C, in the “on” position, when lever 27 is pressed plate 37 moves in the y direction so that hole 37A is directly under container 18 allowing pharmaceutical composition 12 to issue.

Preferably, each actuation of lever 27 causes a single drop 28 (FIg. 1C) of pharmaceutical composition 12 to be issued.

When a dose of several drops is required, lever 27 is actuated several times, each time causing a single drop to be issued. It will be appreciated that lever 27 may be spring operated or otherwise biased, so as to return to its position of FIG. 1A, when released.

As seen in FIG. 1C, single drop 28 of pharmaceutical composition 12 lands on nebulizer 24, preferably including a poezoelectric crystal, which when activated, vibrates so as to turn drop or drops 28 to mist 26.

A fan 13 may be used, also activated by lever 27, to lightly blow mist 26 towards eye 20.

As seen in FIG. 1C, upon the release of lever 27, plate 37 seals container 18.

In accordance with a preferred embodiment of the present invention, device 10 includes an application nozzle 46, having a ring-shaped surface 42, on proximal end 11, with respect to eye 20, for fitting around eye 20.

Application nozzle 46 may be adapted for replacement between uses, allowing it to be disposable, in order to increase hygiene. Alternat5ively, application nozzle 46 may be sterilized between uses, as will be described hereinbelow.

Preferably, nebulizer 24 is adapted for variable frequency and (or) intensity, so as to vary a mean diameter size of droplets of mist 26. In accordance with the present invention, the mean diameter size of mist 26 is less than 10 microns, less than 8 microns, less than 5 microns, less than 3microns and even less than 1 micron.

It will be appreciated that small sized droplets are advantageous for ophthalmic administration due to the reduction of the initiation of the blinking reflex.

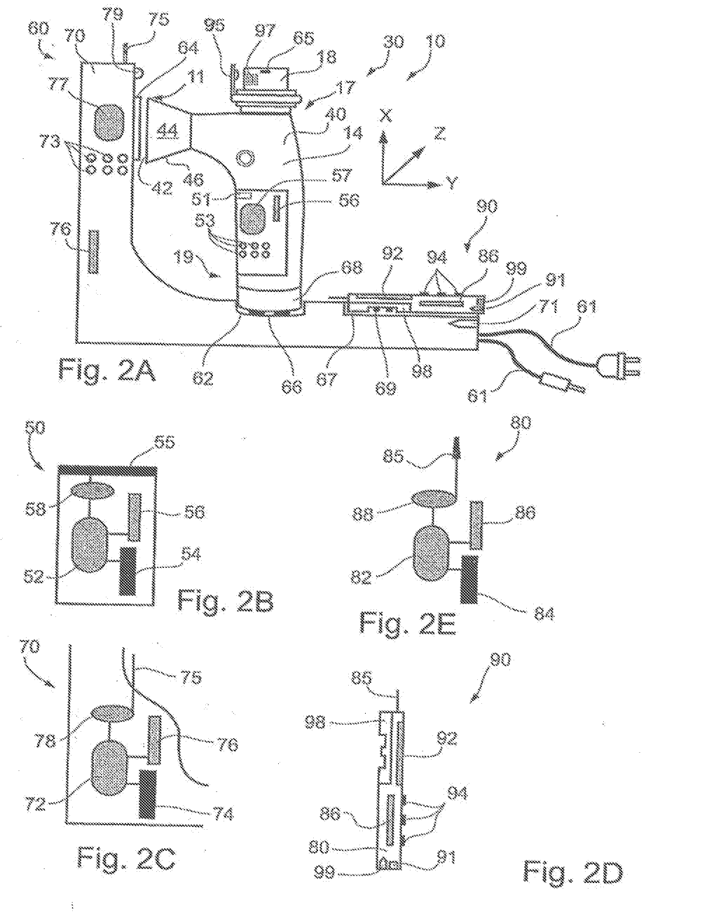

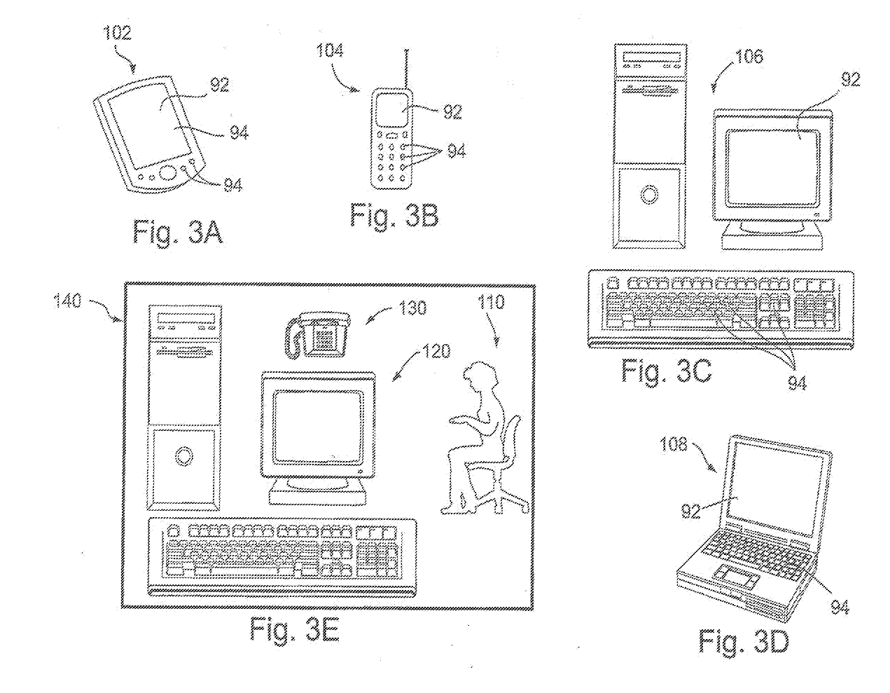

As seen in FIGS. 1F-1H, actuating mechanism 22 may be motor operated. For example, a motor shaft 33 attached to a cam 31 may be used, rotatable in the direction of arrow 35 (FIG. 1H), and pressed against container 18. Container 18 may be held in place by an anchoring device 39. Cam 31 squeezes container 18 as it rotates, causing the issue of one drop 28 with each cycle. the number of cycles of motor shaft 33 determines the number of drops 28. Additionally, the rotation of motor shaft 33 may be controlled by a computerized device, as will be discussed hereinbelow, in conjunction with FIGS. 2A-2E and 3A-3E.

Alternatively, the motor may be activated by a switch 59, located on device 10.

A sensor 34, preferably, an optical sensor, adapted to detect light reflected from eye 20 may be used, in order to determine that eye 20 is open, prior to the activation of device 10.

It will be appreciated that a computerized device, as discussed hereinbelow, in conjunction with FIGS. 2A-2E and 3A-3E, may be used to automatically activate device 10, when sensor 34 senses a reflection from eye 20.

Referring further to the drawings, reference is made to FIGS. 2A-2E, which schematically illustrate an embodiment of a device 10 of the present invention as part of a system 30, comprising, device 10, a complementary stand 60, and a remote control 90, in accordance with a preferred embodiment of the present invention.

As seen in FIG. 2A, device 10, includes a nebulizer portio mist-genertor head 40, which includes nebulizer 24 and actuating mechanism 22 (FIGS. 1A-1D). Additionally, device 10 may include a computerized device 50, and a power source such as a rechargeable battery 68. Complementary stand 60 preferably includes a receptor 62, for receiving body 14 of device 10, and a UV source 74, arranged so that when body 14 is received in receptor 62, UV source 64 ios aimed at ring-shaped surface 42, of application nozzle 46, for effecting sterilization thereof.

UV source 64 may be for example, UV laser diodes, whether stationary rotating, or sweeping.

Additionally, UV source 64 may be aimed at internal surface 44 of application nozzle 46, for sterilizing it as well.

Additionally, UV source 64 may be aimed at the internal components of mist-generator head 40, for sterilizing them as well. These may include mist generator 24, cap 37, the external surfaces of threads 16 and 15, sensor 34, fan 13 and related surfaces.

It will be appreciated that sterilization is performed without affecting pharmaceutical composition 12 in container 18. In accordance with one preferred embodiment, sterilization is performed after container 18 is removed from device 10. Adapter 16 (FIG. 1A) may then be plugged with a plug 65, for keeping nebulizer 24 dust free.

Alternatively, container 18 is arranged so that geometrically, it is not in the line of sight of the UV radiation.

Preferably, complementary stand 60 cinoruses a recgargubg devuce 66 for charging battery 68, which powers nebulizer 24 and preferably also fan 13 and where used, optical sensor 34. Where actuating mechanism 22 is motorized, battery 68 may power the motor as well.

Further in accordance with the present invention, device 10 may include a computerized device 50.

As seen in FIGS. 2A and 2B, illustrating external and internal features, respectively, computerized device 50 includes a processor 52, which preferably includes a control unit, a logic unit (ALU) and memory. Additionally, computerized device 50 may include a fixed data storage device 54, such as a hard disk, and a drive 56 for reading from and (or) writing to a removable data storage device 54, sch as a hard disk, and a drive 56 for reading from and (or) writing to a removable data storage device, such as a minidisk, control buttons 53, preferably, a display screen 57, and possibly also a USB connector 51. Computerized device 50 may also include a transceiver 58, and preferably also an antenna 55, for RF or IR communication, for example, using Bluetooth prtocol. It will be appreciated that a receiver and (or) a transmitter may be used, in place of transceiver 58.

Additionally, device 10 may include a scanner 95, located, for example, on holding structure 21, adapted for reading a bar code 97, so as to identify the medication prior to its application. Alternatively, scanner 95 is adapted for reading letters, so as to identify the medication prior to its application.

Preferably, actuating mechanism 22 is motorized and computerized device 50 may be programmed to turn motor shaft 33 a predetermined number of times, for each application. In this manner, computerized device 50 may control the application dose. Additionally, the programming may be carried out via control buttons 53, via a remote control, or by the insertion of a removable data storage device, such as a minidisk to drive 56.

It will be further appreciated that data related to the application regimen, for example, the application substance, the dosage that was applied, the oeprating parameters of mist generator 24, the frequency of applications, and the exact timing of each application may be stored on fixed data storage device 54, or on a removable data storage device, associated with drive 56. Additionally or alternatively, the data may be displayed on screen 57. Additionally or alternatively, the data may be forwarded to a computer or to a medical center, as will be described hereinbelow, in conjunction with FIGS. 2A-2E and 3A-3E, for follow-up. Tje data may be important for reviewing the effectiveness of the application substance, and to ensure compliance.

Additionally, computerized device 50 may call, beep, or otherwise remind a user of a scheduled application time, to ensure that the user does not miss an applicaation, for example, due to forgetfulness.

In accordance with the present invention, complementary stand 60 may include a computerized device 70, whucg may work in tandem with computerized device4 50, or replace it, partially or completely.

As seen in FIGS. 2A and 2C, illustrating external and internal features, respectively, computerized device 70 may include a fixed data storage device 74, such as a hard disk, and a drive 76 for reading from and (or) writing to a removable data storage device, such as a minidisk, control buttons 73, preferably a display screen 77, and possibly also, a USB connector 71. Computerized device 70 may also include a transceiver 78, and preferably also an antenna 75, for RF or IR communication, for example, using BlueTooth protocol.

Computerized device 70 may be used to control device 10 and to store and (or) display data relating to the application. Additionally or alternatively, computerized device may be used to control and monitor UV source 64.

Additionally, device 10 may include a scanner 79, for example, in place of scanner 95 of device 10. Scanner 79 may be adapted for reading bar codes or letters, so as to identify the medication bottle prior to its application.

A cable 61 connects complementary stand 60 with the grid.

Additionally, a cable 63 may provide internet and (or) phone connections.

Additionally or alternatively, complementary stand 60 may include remote control unit 90, which may work in tandem with computerized devices 50 and 70 or replace them, partially or completely.

As seen in FIGS. 2A, 2D, and 2E, remote control unit 90 is preferably adapted to fit into a receptor 67 of complementary stand 60, and may recharge its battery 98, via a recharging device 69. Preferably, remote control unit 90 includes a computerized device 80, which may include a fixed data storage device 84, such as a hard disk, and a drive 86 for reacing from and (or) writing to a removable data storage device, such as a minidisk, control buttons 94, preferably, a display screen 92 and possibly also a USB connector 91. Remote control unit 90 may also include a transceiver 88, and preferably also an antenna 85, for RF or IR communication, for example, using BlueTooth protocol.

Remote control unit 90 may be used to control device 10 and to store and (or) display data relating to the application. Additionally or alternatively, remote control unit 90 may be used to control and monitor UV source 64.

Additionally, remote control unit 90 may include a scanner 99, for example, in place of scanner 95 of device 10, and (or) in place of scanner 79 of complementary stand 60. Scanner 99 may be adapted for reading bar codes or letters, so as to identify the medication bottle prior to its application.

It will be appreciated that data relating to the application regimen may be stored on fixed data storage devices 74 or 84, or on removable data storage devices, associated with drives 76 or 86. Additionally or alternatively, the data may be displayed on screen 77 or on screen 92. Additionally or alternatively, the data may be forwarded to another computer or to a medical clinic.

Additionally, computerized devices 70 or 80 may call, beep, or otherwise remind a user of a schedule application time, to ensure that the user does not miss an application, for example, due to forgetfulness.

It will be appreciated that device 10 need not be positioned on complementary stand 60 constantly; rather, it may be carried, for example, in a pocket, or in a purse, for use during the day.

Referring further to the drawings, FIGS. 3A-3E schematically illustrate additional computerized devices, which may be used together with ophthalmic delivery system 30 (FIG. 2A), in accordance with the present invention.

as seen in FIG. 3A, a palmtop 102 may be used, in place of, or in addition to remote control unit 90. Additionally, or alternatively, as seen in FIG. 3B, a PDA 104 may be used. Additionally, or alternatively, as seen in FIG. 3C, a personal computer 106 having a modem may be used. Addtionally, or alternatively, as seen in FIG. 3D, a laptop 108 may be used.

These may be used for remotely controlling ophthalmic delivery system 30, for receiving data from ophthalmic delivery system 30, and for storing, displaying, and (or) analyzing the data. Additionally or alternatively, these may be used for forwarding data from ophthalmic delivery system 30 to a medical clinic 140, which preferably includes an attendant 110, a computer 120 and a phone 130. It will be appreciated that medical clinic 140 may be a clinic on the go, for example, of a doctor, his mobile phone, and his laptop. Medical clinic 140 may be used for follow-up of the medical treatment, for example, in order to evaluate the effectiveness of a particular drug, and in order to verify compliance.

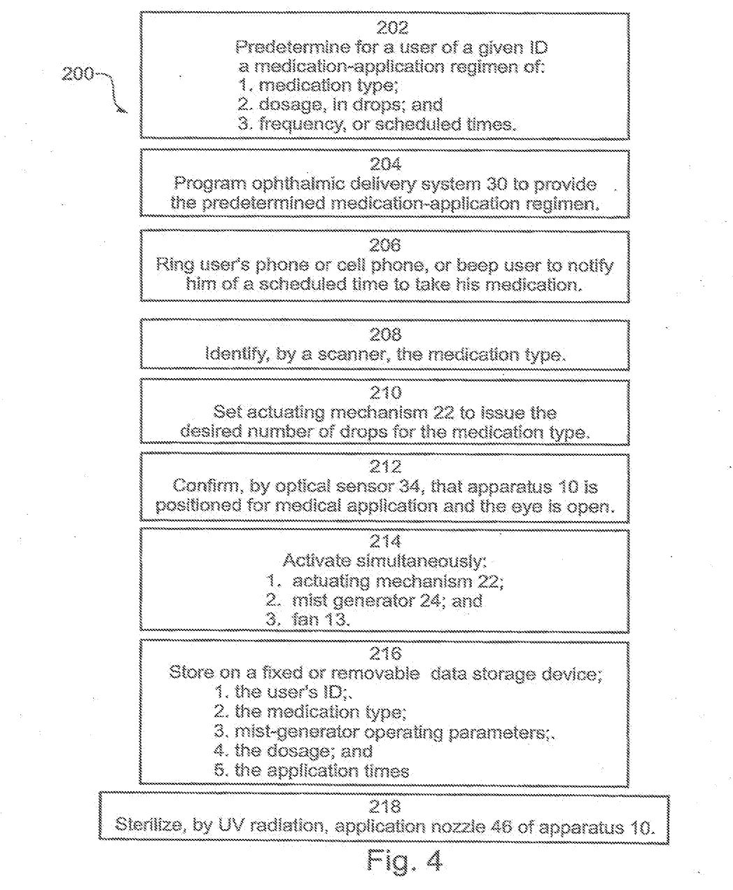

Referring further to the drawings, FIG. 4 is a flowchart of an embodiment of a method 200 of using the ophthalmic delivery system of the present invention:

In a box 202: predetermine for a user of a given ID, a medication-application regimen of: 1. medication type; 2. dosage, in drops; and 3. frequency, or schedule times.

In a box 204: program ophthalmic delivery system 30 to provide the predetermined medication-application regimen.

In a box 206: ring user's phone or cell phone, or beep user, to notify him of a scheduled time to take his medication.

In a box 208: identify, by a scanner, the medication type.

In a box 210: set actuating mechanism 22 to issue the desired number of drops for the medication type.

In a box 212: confirm, by optical sensor 34, that device 10 is position for medical application and the eye is open.

In a box 214: activate simultaneously; 1. actuating mechanism 22; 2. nebulizer 24; and 3. fan 13.

In a box 216: store on a fixed or removable data storage device: 1. the user's ID; 2. the medication type; 3. mist-generator operating parameters; 4. the dosage; and 5. the application times.

In a box 220: sterilize, by UV radiation application nozzle 46 of device 10

It will be appreciated that only a portion of the steps described may be employed. It will be further appreciated that other methods that utilize the features of ophthalmic delivery system 30 may similarly be used.

As described above and with reference to device 10 in fIGS. 1A-1H and 2A-2D it is advantageous to direct a mist towards and eye only when the eye is open. Thus an addditional aspect of the present invention relates to a device for ophthalmic administration comprising a nebulizer, a mist director to to direct a generated mist at an eye, an eye-state detector to detect if an eye is open or closed, and a switch associated with both the eye-state detector and the mist director to direct mist at the eye only when open. Thus, in embodiments of the present invention there is provided a device (e.g., 10) for ophthalmic administration of a composition (preferably a pharmaceutical composition), comprising; a) a misting unit (e.g., 40) including i) a nebulizer (e.g., 24), configured to generate a mist from a composition (e.g., 12); ii) a mist director (e.g., 13), configured to direct mist generated by the nebulizer at an eye; b) an eye-state detector (e.g., 34), configured to detect if the eye is open or shut; and c) a switch functionally associated with the misting unit and with the eye-state detector having at least two states, an “ON” state wherein a mist is directed at the eye and an “OFF” state wherein a mist is not directed at the eye.

In embodiments of the present invention, the switch sets to the “ON” state when the eye-state detector detects that the eye is open and/or the switch sets to the “OFF” state when the eye-state detector detects that the eye is shut.

In embodiments, the mist director is a physical component distinct from the nebulizer. In embodiments, the mist director is not a physical component distinct from the nebulizer. For example, in embodiment the mist director is simply a tube or passageway between the nebulizer and the eye.

In embodiments direction of the mist to the eye is performed by controlling the nebulizer. In such embodiments, the nebulizer (e.g., 24) is deactivated when the switch is set to the “OFF” state and the nebulizer is activated when the switch is set to the “ON” state.

In embodiments direction of the mist to the eye is performed by controlling a valve or similar component, not depicted in the figures above. In such embodiments, the misting unit further comprises a valve functionally associated with the mist director, and the valve is configured to close when the switch is set to the “OFF” state and the valve configured to open when the switch is set to the “ON” state.

In embodiments direction of the mist to the eye is performed by controlling a blower, e.g., a fan 13, compressor other such component. In such embodiments, the misting unit further comprises a blower functionally associated with the mist director, the blower being deactivated when the switch is set to the “OFF” state and the blower being activated when the switch is set to the “ON” state.

Many different technologies and components are useful in implementing an eye state detector, including cameras associated with an image processing unit to identify when an eye is open or shut, for example by identifying the iris, the pupil, eyelashes, eyelid or distinct color, for example of the sclera. Due to the fact that the anterior surfaces of the sclera and cornea are reflective, detection of reflection such as specular reflection from the anterior portion of the eye, in embodiments of the present invention the eye state detector is configured to detect light reflecting from the surface of an anterior portion of an open eye. One method of implementing such a reflector is with the use of a light-emitting diode (e.g., projecting visible or near-infrared light) to illuminate the eye surface and a ligh detecting diode (e.g., a silicon PIN photodiode) configured to detect light reflected from the eye surface.

Embodiments of a device of the present invention include an easily replaceable contact surface, allowing the contact surface to be disposable. Specifically, a nozzle 46 is configured to be easily attachable to and detachable from the rest of the device. Between every administration, a user detaches the used nozzle and attaches a clean (preferably new or sterile) nozzle. The used nozzle is discarded or cleaned and/or sterilized.

An additional aspect of the present invention relates to a device for ophthalmic administration that is self-sterilizing. As describe above and with reference to device 10 in FIGS. 1A-1H and 2A-2D it is advantageous to sterilize an ophthalmic delivery device between individual administrations. A device that is quick and easy to serilize allows high throughput and safe ophthalmic administration of a pharmaceutical composition, for example in a hospital or clinic where many patients may be treated with one device, or in high-throughput situations, for example when desired to treat a population for an epidemic or endemic condition or to inoculate or immunize a population. For example, the use of an embodiment of a pharmaceutical composition of the present invention including an antibody as an API together with an easily sterilizable device, allows a large population to be immunized against an illness.

Thus, an addtional aspect of the present invention relates to a device for ophthalmic administration of a pharmaceutical composition to an eye of a subject, comprising: a) a contact component with a contact surface (e.g., 42), the contact surface configured to contact a portion of the body of the subject during the administration (e.g., the eye, the area around the eye, the eyelids); and b) a reversibly actuatable radiation-source (e.g., 64), configured to irradiate the contact surface with sterilizing radiation. As noted above, such a radiation-source is advantageously configured to irradiate other components of a respective device also.

An suitable sterilizing radiation is useful for implementing the sterilizing aspect teachings of the present invention. Embodiments of the present invention include sterilizing radiation comprising radiation selected from the group consisting of coherent radiation, incoherent radiation, microwave radiation, infrared radiation and ultraviolet radiation.

In embodiments, such as device 10 as depicted in FIG. 2A, the contact component is an integral element of a first unit of the device (e.g., 10) and the radiation source is an integral element of a second unit of the device (e.g., 60), wherein the first unit and the second unit are physically distinct. As described above, when activated the radiation source sterilizes the contact component by projecting sterilizing radiation at the contact surface. In such embodiments, it is advantageous that the first unit has a power source such as a rechargeable battery, and the second unit includes a recharger, so that the first unit is sterilized during recharging, as described abov e.

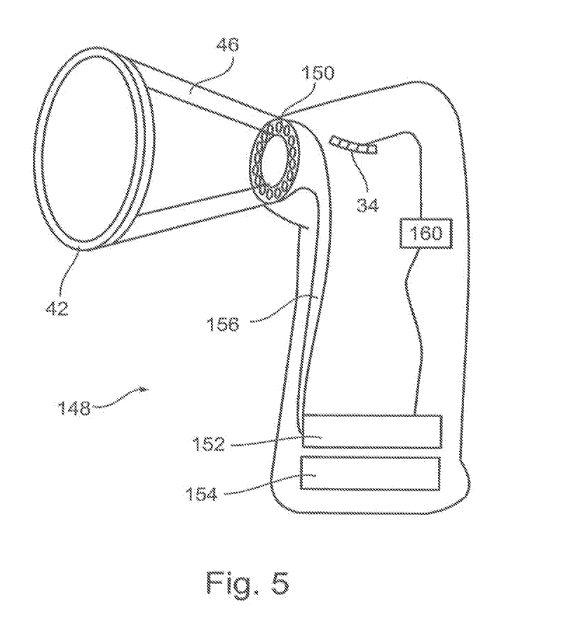

In embodiments of the present invention, both a contact component and a radiation source are both integral elements of a single unit of the device. Such an embodiment of the present invention 148 is depicted in FIG. 5. In FIG. 5 is depicted a nozzle 46 with ring-shaped contact surface 46 at a distal end and a ring of sterilizing radiation sources 150. Sources 150 are functionally associated with a UV-lamp 152 (Spectronics Corporation, Westbury, N.Y., USA) powered by a battery 154. When activated, UV-lamp 152 produces sterilizing radiation that is transported through optical fibers 156 to sources 150. Nozzle 46 is transparent to the radiation produced by UV-lamp 152 and is configured to act as a wave guide for the UV light, guiding the light to contact surface 42 to sterilize contact surface 142.

In embodiments of the present invention, a radiation source is user-actuated, for example by the press of a button when desired.

In embodiments of the present invention, such as depicted in FIG. 2A, the radiation-source is automatically activated: when device 10 is placed in stand 60, UV-source 64 is activated.

In embodiments of the present invention, such as depicted in FIG. 5, the radiation source is autonomously activated, that is the device is configured to activate a respective radiation source when needed. In FIG. 5, device 148 is provided with a sensor 34 as described above functionally associated with logic unit 160 that is configured to activate UV-lamp 152. When device 148 is actuated to dispense a pharmaceutical composition, logic unit 160 interrogates sensor 34 whether an eye (or any object) is positioned in front of nozzle 46. If yes, logic unit 160 waits until the front of nozzle 46 is clear. When nozzle 46 is clear, logic unit 160 activates UV-lamp 152 to sterilize contact surface 42. During sterilization, logic unit 160 monitors sensor 34

In such a way, device 148 is provided with a fail-safe mechanism preventing inadvertent irradiation of an eye that may cause damage, making such a device exceptionally useful for high-throughput situations, even when an operator is not highly skilled.

Embodiments of a device of the present invention are equipped with a timer to ensure that irradiation is performed for a sufficiently long time for effective sterilization.

An additional aspect of the present invention is a method and a device for increasing the bioavailability of an ophthalmically administered API, whether for systemic or local delivery, whether through the conjunctiva, sclera, cornea or other route, by vibrating the eyelid subsequent to the ophthalmic administration.

As discussed herein, a problem with ophthalmic administration is that it often a significant portion of an administered API does not penetrate into the body. A method of the present invention to increase the bioavailability of an ophthalmically administered API comprises a) contacting a composition, preferably a pharmaceutical composition, with a posterior section of an eye; b) shutting the eye with a respective eyelid; and c) vibrating the eyelid. As a result of the vibrations, the API in the pharmaceutical composition penetrates the ocular tissue more easily and quckly, increasing the bioavailability thereof.

Any suitable method of contacting the composition is suitable, including insilling eyedrops with an eye dropper or other device, spraying, or with a mist, for example using an embodiment of a device of the present invention.

In an embodiment of the present invention, the eyelid is vibrated using a vibrating physical component, see below.

Various frequencies of vibration are effective in increasing the bioavailability of an ophthalmically administered API according to the present invention. In embodiments of the present invention the vibration are include sonic and/or ultrasonic frequencies, such as frequencies of between about 10 Hz and 100 mHz, frequencies of no less than about 1 kHz, frequencies of no less than about 10 kHz or frequencies of no less than about 1 mHz.

Generally any period of time of vibration is effective in increasing the bioavailability of an ophthalmically administered API according to the present invention to some extent. That said, in embodiments of the present invention the vibrating is for at least 10 seconds, for at least 30 seconds or even for at least 60 seconds.

Useful for vibrating an eyelid in accordance with the teachings of the present invention is an eyelid vibrating device of the present invention.

An eyelid vibrating device of the present invention is a device for increasing the bioavailability of an ophthalmically administered API in a pharmaceutical composition, comprising: a) an eyelid contact component, configured to physically contact an eyelid of an eye and maintain the eyelid in a shut position; and b) a vibration generator configured to generate vibrations and transfer the vibrations to the eyelid contact component.

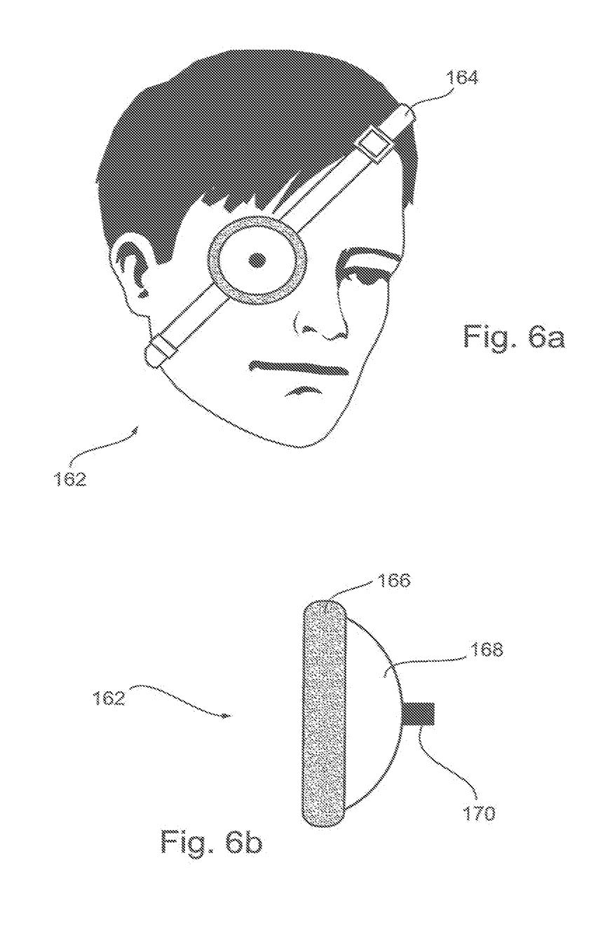

An embodiment of an eyelid vibrating device 162, substantially an eye patch, of the present invention is depicted in FIG. 6A fixed to the head of a person and in FIG 6B in side view. Device 162 is configured with a head band 164 to act as a holder to hold a contact component 166 (a silicon rubber disk) against the shut eyelid of a person.

Contact component 166 is a soft silicon rubber disk configured to effectively transfer vibrations generated by vibratioen generator 168 to the eyelid of a person with damage such as chafing or scratching. In embodiments of the present invention, a contact component is a sack or bag holding a liquid (e.g., a saturated saline solution) to transfer vibrations.

Vibration generator 168 is functionally associated with contact component 166 and includes a piezoelectric crystal (not depicted) as a vibration generator, a power source (not depicted) and a switch 170. In embodiments of the present invention, instead of or in addition to a piezoelectric crystal other vibration generating components are use. For example, in embodiments a vibrating diaphragm (such as in an audio speaker) is used as a vibrating generating component of a vibration generator.

When switch 170 is set to an “ON” state, vibration generator 168 generates vibrations (of a desired frequency, as described above, in embodiments comprising ultrasonic or sonic frequencies) that are transferred to the eyelid of a person through contact component 166.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non-limiting fashio.

### Nebulization Device

Ophthalmic administration of pharmaceutical compositions as a mist in accordance with embodiments of the present invention was performed with a nebulization device constructed by the inventor.

### Materials

Materials and reagents were purchased from Sigma Chemical Company (St. Louis, Mo., USA).

Animal studies were carried out on Lewis female rats (6-8 weeks of age) from Harlan Laboratories Inc. (Rehovot, Israel). In some cases as noted below, the rats were anesthetized with ketamin/xylazine using a 27 G cannula through the cisterna magna.

ELISA tests were performed using commercially available ELISA kits from Assay Designs (Ann Arbor, Mich., USA) in accordance with the manufacturer's instructions in the usual way in conjunction with a BioTek EL-310 plate reader (Cambridge Scientific Products, Cambridge, Mass., USA).

Pharmaceutical compositions were administered by instillation in the eyes of rats or administered as a mist using the nebulization device described above at a rate of 10 μl minute⁻¹. Subsequent to administration and a waiting time, the rats were sacrificed.

Subsequent to sacrifice, serum, cerebrospinal fluid (CSF, 50-100 μl), retina, optic nerve, sclera, and aqueous humor (AH) of an animal were harvested. The retina, optic nerve, and sclera of each animal was individually homogenized in 120 ul assay buffer on ice and centrifuged. The serum, CSF, AH and the homogenized retina, optic nerve and sclera were assayed for the presence of leptin using ELISA.

Data below is expressed as pg ml⁻¹ for serum, CSF and AH and as pg mg⁻¹ for tissue (determined using the Lowry method).

### Example 1

### Delivery of Middle Sized Protein by Ophthalmic Administration

A first leptic composition was prepared including 0.6 mg ml⁻¹ leptin (16 kDa) in a standard phosphate buffered saline (PBS) ophthalmic vehicle having a pH of 7.4.

A second leptin/saponin compositions was prepared including 0.6 mg ml⁻¹ leptin and 1% saponin as a penetration enhancer in a standard phosphate buffered saline (PBS) ophthalmic vehicle having a pH of 7.4.

### a. Delivery of Middle Sized Protein to the Retina

The first leptin composition was administered for 2 minutes as a mist to the eyes of a first group of rats and 1 drop (about 30 μl) instilled into the eyes of a second group of rats. The rats of the two groups were sacrificed 10 minutes after administration. The levels of leptin in the retina and in the AH were compared to those of a control group. the results are depicted in FIGS. 7A and 7B. In FIG. 7A it is seen that both instillation and administration as a mist deliver similar levels of leptin to the retina. In FIG. 7B it is seen that administration by instillation delivers significantly more leptin to the AH that does administration as a mist, suggesting that the delivery of leptin to the posterior portion of the eye occurs by a route different than that of instillation.

The second leptin/saponin composition was administered for 2 minutes as a mist to the eyes of a first group of rats and 1 drop (about 30 μl) instilled into the eyes of a second group of rats. the rats of the two groups were sacrificed 20 minutes after administration. The retina leptin levels were compared to those of a control group. The results are depicted in FIG. 7C. In FIG. 7C it is seen that both administration by instillation and as a mist deliver similar levels of leptin to the retina in the presence of 1% saponin.

### b. Delivery of Middle Sized Protein to the Central Nervous System

The first leptin composition was administered for 2 minutes as a mist to the eyes of a first group of rats and 1 drop (about 30 μl) instilled into the eyes of a second group of rats. the rats of the two groups were sacrificed 10 minutes after administration. The levels of leptin in the CSF were compared to those of a control group. The results are depicted in FIG. 8A. In FIG. 8A it is seen that neither instillation nor administration as a mist deliver a statistically significant amount of leptin to the CSF.

The second leptin/saponin composition was administered for 2 minutes as a mist to the eyes of a first group of rats and 1 drop (about 30 μl) instilled into the eyes of a second group of rats. The rats of both groups were sacrificed 10 minutes and 20 minutes after administration. The retina leptin levels were compared to those of a control group. The results are depicted in FIG. 8B. In FIG. 8B it is seen that whereas administration by instillation did not deliver a statistically significant amount of leptin to the CSF, administration as a mist did deliver a statistically significan amount of leptin to the CSF.

Thus, it is demonstrated that ophthalmic administration of a pharmaceutical composition of an API such as a middle-sized protein with a penetration enhancer such as saponin as a mist is effective in delivery of the API to the central nervous system.

### c. Route of Delivery of Middle Sized Protein to the Central Nervous System

The second leptin/saponin composition was administered for a period of 15 second, 30 seconds, 1 minute and 2 minutes as a mist to the eyes of groups of rats. The rats were sacrificed 10 minutes after administration. The levels of leptin in the optic nerve and the sclera were compared to those of a control group. The results for accumulation of leptin in the optic nerve are depicted in the table of FIG. 9A in units of pg μg⁻¹, where “low” indicates below the lower limit of detection of the ELISA assay. In FIG. 9A it is seen that 2 minutes were reqired for delivery of a detectable amount of leptin to the optic nerve. The results for accumulation of leptin in the sclera are depicted in FIG. 9B in units of pg μg⁻¹. In FIG. 9B it is seen that 2 minutes were required for delivery of a significant amount of leptin to the sclera.

It should be noted that the leptin concentration in both the optic nerve and sclera was markedly higher than in the the retina. Taken together with the results depicted in FIGS. 9A and 9B, together with the absence of leptin in the AH (FIG. 7B) suggest that protein delivery when administered as a mist is preferentially through the sclera and apparently through a different mechanism then when administered by instillation.

### d. Effect of Continuous Administration of a Protein

The second leptin composition was administered for 2 minutes as a mist to the eyes of a first group of rats and 1 drop (about 30 μl) instilled into the eyes of a second group of rats. The rats of both groups were sacrificed 5 or 10 minutes after administration. A third group of rats was anesthetized and the second leptin composition administered continuously for 10 minutes as a mist to the eyes of rats and the rats were sacrificed after the 10 minutes. A fourth group of rats was anesthetized and drops of the second leptin composition instilled over a period of 10 minutes so as to ensure a continuous supply of the composition in the eye and the rats were sacrificed after the 10 minutes.

Because of the low number of rats in each group, statistical analysis could not be performed.

The retina leptin levels after the full 10 minutes of administration by both methods were compared to those of a control group. The results are depicted in FIG. 10A. In FIG. 10A it is seen that continuous administration for 10 minutes of composition both by instillation and by administration delivers significant amounts of leptin to the retina in the presence of 1% saponin.

The sclera leptin levels of rats sacrificed 5 or 10 minutes after either administration of 1 drop by instillation or 2 minutes administration as a mist of the composition were compared to those of a control group. The results are depicted in FIG. 10B. In FIG. 10B it is seen that both instillation and administration as a mist deliver significant amounts of leptin to the sclera in the presence of 1% saponin.

The optic nerve leptin levels after the full 10 minutes of administration by both methods were compared to those found in of rats sacrificed after 5 or 10 minutes subsequent to either administration by instillation of a drop or by 2 minutes administration as a mist of the composition to those of a control group. The results are depicted in FIG. 10C. In FIG. 10A it is seen that continuous administration for 10 minutes of composition both by instillation and by administration as a mist delivers signifcant amounts of leptin to the optic nerve in the presence of 1% saponin.

It is thus seen that continuous administration both by instillation and by administration as mist delivers significant amounts of leptin to the retina, sclera and optic nerve in the presence of 1% saponin.

### e. Systemic Delivery of Middle Sized Protein

The first leptin composition was administered for 2 minutes as a mist to the eyes of a first group of rats and 1 drop (about 30 μl) instilled into the eyes of a second group of rats. The rats of both groups were sacrificed 10 minutes after administration. The serum levels of leptin were compared to those of a control group. The results are depicted in FIG. 11A. In FIG. 11A it is seen that instillation provides systemic delivery of a leptin but administration as a mist does not provide significant systemic deliver.

The second leptin/saponin composition was administered for 2 minutes as a mist to the eyes of a first group of rats and 1 drop (about 30 μl) instilled into the eyes of a second group of rats. Rats of both groups were sacrificed after 10 and 20 minutes. The serum leptin levels were compared to those of a control group. The results are depicted in FIG. 11B. In FIG. 11B it is seen that neither instillation nor administration as a mist deliver provide significant systemic delivery of leptin in the presence of 1% saponin.

To first group of anesthetized rats the second leptin composition was administered continuously for 10 minutes as a mist to the eyes and the rats sacrificed after the 10 minutes. To a second group of anesthetized rats were administered drops of the second leptin composition by instillation over a period of 10 minutes so as to ensure a continuous supply of the composition in the eye and the rats sacrificed after the 10 minutes. The results are depicted in FIG. 11C. In FIG. 11C it is seen that instillation provides systemic delivery of a leptin but administration as a mist does not provide significant systemic deliver.

Thus is is demonstrated that ophthalmic administration of a pharmaceutical composition of an API such as a middle-sized protein as a mist is effective in selective delivery of the API to the eye and to the central nervous system.

### f. Irritation Effect of Penetration Enhancers

Throughout the studies above it was noted that, as expected, rats to which a composition including 1% saponin was administered by instillation into the eyes instilled exhibited marked irritation, see FIG. 12A.

In contrast and unexpectedly, rats to which a composition including 1% saponin was administered as a mist exhibited no signs of irritation.

Thus, it is demonstrated that ophthalmic administration of a pharmaceutical composition including an irritant penetration enhancer as a mist is possible without causing eye irritation.

### Example 2

### Delivery of an Antibody by Ophthalmic Administration to the CNS

A composition containing mouse 2.5 μg ml⁻¹ IgGl and 1% saponin in a standard phosphate buffered saline (PBS) ophthalmic vehicle having a pH of 7.4 was prepared.

Using the nebulizer device described above, 20 μl of the IgGl composition was administered over a period of 2 minutes to each eye of 30 rats making up a first group of rats. Using a nebulizer device, 50 μl of the IgGl solution was administered over a period of 5 minutes to each eye of 30 rats making up a second group of rats. Using a nebulizer device, 100 μl of the IgGl solution was administered over a period of 10 minutes to each eye of 30 rats making up a third group of rats. Using a nebulizer device, 100 μl of the IgGl solution was administered over a period of 10 minutes to each eye of 30 rats making up a fourth group of rats. A group of 30 untreated rats make up a control group.

The rats of the first, second and third group were sacrificed 10 minutes after initiation of nebulization. The rats of the fourth group were sacrificed 20 minutes after initiation of nebulization. The control group was also sacrificed.

From each rat, the two retina and the two optic nerves were harvested, homogenized and the supernatant assayed for the presence of mouse IgGl in the usual way using ELISA.

No significant amount of mouse IgGl was found in any of the retina.

There was a significant accumulation of mouse IgGl in the optic nerves of the rats, see FIG. 13. The greatest levels of mouse IgGl were found in the rats to which the composition was continuously administered for ten minutes and then allowed an additional ten minutes to reach the posterior section of the eye.

As the mouse IgGl accumulated in the optic nerve, and as the optic nerve is surrounded by CSF and is directly connected to the brain, it is expected that large proteins, such as antibodies such as IgGl are deliverable to the central nervous system using the teachings of the present invention as demonstrated for leptin.

### Prophetic Example 1

### Administration of GDNF (Glial Derived Neurotrophic Factor)

A composition containing GDNF as an API with 2% benzalkonium chloride in a standard phosphate buffered saline (PBS) ophthalmic vehicle with a pH of 7.4 is prepared and administered as a mist in accordance with the teachings of the present invention to accumulate in the retina, optic nerve, CSF, and brain of a subject, and thus treat conditions of the retina and central nervous (CNS), such as Parkinson's disease, see Sherer, T. B. et al. Exp. Neurol. 2003, 179, 9-16.

### Prophetic Example 2

### Administration of Bevacizumab

Bevacizumab is an inhibitor of Vascular Endothelial Growth Factor (VEGF), a protein that plays an important role in tumor angiogenesis and maintenance of existing tumor vessels. By inhibiting VEGF, Bevacizumab interferes with the blood supply to tumors, a process that is critical to tumor growth and metastasis.

A composition containing Bevacizumab as an API in a standard phosphate buffered saline (PBS) ophthalmic vehicle with a pH of 7.4 is prepared and administered as a mist in accordance with the teachings of the present invention to treat cancers, such as metastatic colon or rectum cancer and also for delivery to the retina and central nervous (CNS) to treat cancers therein.

### Prophetic Example 3

### Administration of Ranibizumab

A composition containing Ranibizumab as an API with 0.5% deoxycholic acid as a penetration enhancer in a standard phosphate buffered saline (PBS) ophthalmic vehicle with a pH of 7.4 is prepared and administered as a mist in accordance with the teachings of the present invention to treat a subject in need thereof.

### Prophetic Example 4

### Administration of Ieredelimumab

A composition containing Ieredelimumab as an API with 0.1% digitonin as a penetration enhancer in a standard phosphate buffered saline (PBS) ophthalmic vehicle with a pH of 7.4 is prepared and administered as a mist in accordance with the teachings of the present invention to treat a subject in need thereof.

### Prophetic Example 5

### Administration of Dipivefrin HCl

To a commercially available pharmaceutical composition including 0.15% Dipivefrin HCl in an ophthalmically acceptable carrier (comprising benzalkonium chloride (0.05 mg/ml) as a preservative, edetate disodium, sodium chloride, hydrochloric acid to adjust pH to about 2.5-3.5, and purified water) such as Propine® is added 1% fusidic acid to provide an ophthalmic composition of the present invention.

1 microliter of the ophthalmic composition of the present invention is applied twice daily as a mist using a device of the present invention to a subject suffering from ocular hypertension. Marked reduction of the intraocular pressure is observed.

### Prophetic Example 6

### Administration of Apraclonidine HCl

To a commercially available pharmaceutical composition including 5.75 mg/ml Apraclonidine HCl (equivalent to 0.5% Apraclonidine) in an ophthalmically acceptable carrier (comprising benzalkonium chloride (0.01 mg/ml) as a preservative, sodium chloride, sodium acetate, sodium hydroxide and/or hydrochloric acid (to adjust pH to about 2.5-3.5) and purified water) such as Iopidine® is added 1% sodium deoxycholate to provide and ophthalmic composition of the present invention.

1 microliter of the ophthalmic composition of the present invention is applied three times daily as a mist using a device of the present invention to a subject suffering from ocular hypertension. Marked reduction of the intraocular pressure is observed.

### Prophetic Example 7

### Administration of Dapiprazole

To a commercially available pharmaceutical composition including 0.5%; Dapiprazole in an ophthalmically acceptable carrier (comprising benzalkonium chloride (0.01%) as a preservative, mannitol (2%), sodium chloride, hydroxypropyl methylcellulose (0.4%), edetate sodium (0.01%), sodium phosphate dibasic, sodium phosphate monobasic and purified water having a pH of 6.6) such as Rev-Eyes ® is added 2% fusidate, to provide an ophthalmic composition of the present invention.

2 microliter of the ophthalmic composition of the present invention is administered to the eye of a subject having diagnostic mydriasis. After 5 minutes, an additional 2 microliters of the ophthalmic composition of the present invention is administered. Marked reduction of pupil size is observed.

### Prophetic Example 8

### Administration of Dorzolamide

To a commercially available pharmaceutical composition including 2% Dorzolamide (22.3 mg/ml of dorzolamide hydrochloride) in an ophthalmically, acceptable carrier (comprising benzalkonium chloride (0.0075%) as a preservative, hydroxyethyl cellulose, mannitol, sodium citrate dihydrate, sodium hydroxide (to adjust pH to 5.6) and purified water such as Trusopt® is added 2% ammonium glycyrrhizide as a penetration enhancer to provide an ophthalmic composition of the present invention.

1 microliter of the ophthalmic composition of the present inventio is applied three times daily as a mist using a device of the present invention to a subject suffering from ocular hypertension. Marked reduction of the intraocular pressure is observed.

### Prophetic Example 9

### Administration of Timolol

To a commercially available pharmaceutical composition including 0.25% Timolol (3.4 mg/ml Timolol Maleate) in an ophthalmically acceptable carrier (comprising benzalkonium chloride (0.01%) as a preservative, monobasic and dibasic sodium phosphate, sodium hydroxide (to adjust pH) and purified water such as Timoptic® is added 3% Brij 35 as a penetration enhancer to provide an ophthalmic composition of the present invention.

1 microliter of the ophthalmic composition of the present invention is applied twice daily as a mist using a device of the present invention to a subject suffering from ocular hypertension. Marked reduction of the intraocular pressure is observed.

### Prophetic Example 10

### Administration of Dorzolamide with Timolol

To a commercially available pharmaceutical composition including 2% Dorzolamide (22.3 mg/ml of dorzolamide hydrochloride) and 0.5% Timolol (6.83 mg/ml Timolol Maleate) in an ophthalmically acceptable carrier (comprising benzalkonium chloride (0.0075%) as a preservative, hydroxyethyl cellulose, mannitol, sodium citrate, sodium hydroxide (to adjust pH to 5.6) and purified water such as Cosopt® is added 4% Brij-98 as a penetration enhancer to provde an ophthalmic composition of the present invention.

1 microliter of the ophthalmic composition of the present invention is applied twice daily as a mist using a device of the prewsent invention to a subject suffering from ocular hypertension. Marked reduction of the intraocular pressure is observed.

### Prophetic Example 11

### Administration of Unoprostone Isopropyl

To a commercially available pharmaceutical composition including 0.15% Unoprostone Isopropyl (1.5 mg/ml of Unoprostone Isopropyl) in an ophthalmically acceptable carrier (comprising benzalkonium chloride (0.015%) as a preservative, mannitol, polysorbate 80, edetate disodium, sodium hydroxide or hydrochloric acid (to adjust pH to 5.0-6.5) and purified water such as Rescula® is added 2% cetylpyridium chloride as a penetration enhancer to provide an ophthalmic composition of the present invention.

1 microliter of the ophthalmic composition of the present invention is applied twice times daily as a mist using a device of the present invention to a subject suffering from ocular hypertension. Marked reduction of the intraocular pressure is observed.

### Prophetic Example 12: Administation of Levobunolol

To a commercially available pharmaceutical composition including 0.25% Levobunolol (as Levobunolol HCl) in an ophthalmically acceptable carrier (comprising benzalkonium chloride (0.004%) as a preservative, edetate disodium, polyvinyl alcohol, potasium phosphate monobasic, sodiu chloride, sodium metabisulfite, sodium phosphate dibasic and hydrochloric acid or sodium hydroxide (to adjust pH to 5.5-7.5) and purified water such as Betagan Liquifilm® is added 3% cholic acid as a penetration enhancer to provide an ophthalmic composition of the present invention.

1 microliter of the ophthalmic composition of the present invention is applied twice times daily as a mist using a device of the present invention to a subject suffering from ocular hypertension. Marked reduction of the intraocular pressure is observed.

### Prophetic Example 13

### Administration of Betaxolol

To a commercially available pharmaceutical composition including 0.25% Betaxolol (as 2.8 mg/ml Betaxolol Hcl) in an ophthalmically acceptable carrier (comprising benzalkonium chloride (0.01%) as a preservative, mannitol, Poly(Seyrene-Divinyl Benzene) sulfonic acid, Carbomer 934P, edetate disodium, hydrochloric acid or sodium hydroxide (to adjust pH) and purified water such as Betoptic S®) is added 4% decamethonium bromide as a penetration enhancer to provide an ophthalmic composition of the present invention.

1 microliter of the ophthalmic composition of the present invention is applied twice times daily as a mist using a device of the present invention to a subject suffering from ocular hypertension. Marked reduction of the intraocular prssure is observed.

### Prophetic Example 14

### Administration of Pilocarpine

To a commercially available pharmaceutical composition including 5 mg/ml Pilocarpine as Pilocarpine nitrate in an ophthalmically acceptable carrier (comprising benzalkonium chloride as a preservative, boric acid, potassium chloride, hydroxypropylmethyl cellulose, sodium carbonate, edetate disodium and purified water such as Pilagan Liquifilm® is added 0.5% saponin to provide an ophthalmic composition of the present invention.

1 microliter of the ophthalmic composition of the present invention is applied three times daily as a mist using a device of the present invention to a subject suffering from ocular hypertension. Marked reduction of the intraocular pressure is observed.

### Prophetic Example 15

### Administration of Echothiophate Iodide

To a commercially available pharmaceutical composition including 0.125% Echothiophate Iodide (6.25 mg/ml) in an ophthalmically acceptable carrier (comprising 40 mg/ml potassium acetate, chlorobutanol, mannitol, boric acid and exsiccated sodium phosphate such as Phospholine Iodide®) is added 0.5% saponin to provide an ophthalmic composition of the present invention.

1 microliter of the ophthalmic composition of the present invention is applied twice times daily as a mist using a device of the present invention to a subject suffering from ocular hypertension. Marked reduction of the intraocular pressure is observed.

### Prophetic Example 16

### Administration of Latanoprost

To a commercially available pharmaceutical composition including 0.005% Latanoprost (50 mcg/ml) in an ophthalmically acceptable carrier (comprising benzalkonium chloride (0.02%) as a preservative, Sodium chloride, sodium dihydrogen phosphate monohydrate, disodium hydrogen phosphate anhydrous (to adjust pH to 6.7) and purified water such as Xalatan®) is added 0.5% saponin to provide an ophthalmic composition of the present invention.

1 microliter of the ophthalmic composition of the present invention is applied once a day as a mist using a device of the present invention to a subject suffering from ocular hypertension. Marked reduction of the intraocular pressure is observed.

### Prophetic Example 17

### Administration of Bimatoprost

To a commercially available pharmaceutical composition including 0.03% Bimatoprost (0.3 mg/ml) in an ophthalmically acceptable carrier (comprising benzalkonium chloride (0.05 mg/ml) as a preservative, sodium chloride, dibasic sodium phosphate, citric acid, sodium hydroxide and/or hydrochloric acid (to adjust pH to 6.8-7.8) and purified water such as Lumigan®) is added 0.5% saponin to provide an ophthalmic composition of the present invention.

1 microliter of the ophthalmic composition of the present invention is applied once a day as a mist using a device of the present invention to a subject suffering from ocular hypertension. Marked reduction of the intraocular pressure is observed.

### Prophetic Example 18

### Administration of Fluribrofen Sodium

To a commercially available pharmaceutical composition including 0.03% fluribrofen Sodium (0.3 mg/ml) in an ophthalmically acceptable carrier (comprising thimerosal (0.005%) as a preservative, polyvinyl alcohol 1.4%; edetate disodium; potassium chloride; sodium chloride; sodium citrate; citric acid; hydrochloric acid and/or sodium hydroxide (to adjust pH to 6.0-7.0) and purified water such as Ocufen®) is added 0.5% saponin to provide an ophthalmic composition of the present invention.

1 microliter of the ophthalmic composition of the present invention is applied once every half hour four times starting two hours before surgery in order to effectively inhibit intraoperative miosis.

### Prophetic Example 19

### Administration of Prednisolone Acetate

To a commercially available pharmaceutical composition including 1% Prednisolone Acetate in an ophthalmically acceptable carrier (comprising benzalkonium chloride (0.01 mg/ml) as a preservative, hydroxpropyl methylcellulose 2910, dibasic sodium phosphate, Polysorbate 80, ddetate disodium, glycerin, citric acid and/or sodium hydroxide (to adjust pH) and purified water such as Pred Forte®) is added 0.5% saponin to provide an ophthalmic composition of the present invention.

1 microliter of the ophthalmic composition of the present invention is applied once an hour as a mist using a device of the present invention to a subject suffering from ocular inflammation. Marked reduction of inflammation is observed.

### Prophetic Example 20

### Administration of Dexamethasone

To a commercially available pharmaceutical composition including 0.1% Dexamethasone in an ophthalmically acceptable carrier (comprising benzalkonium chloride (0.01%) as a preservative, sodium chloride, hydroxypropyl methylcellulose, dibasic sodium phosphate, polysorbate 80, edetate disodium, citric acid and/or sodium hydroxide (to adjust pH) and purified water such as Maxidex™) is added 0.5% saponin to provide an ophthalmic composition of the present invention.

1 microliter of the ophthalmic composition of the present invention is applied once an hour as a mist using a device of the present invention to a subject suffering from ocular inflammation. Marked reduction of inflammation is observed.

### Prophetic Example 21

### Administration of Triamcinolone Acetonide

A composition including an appropriate concentration of Triamcinolone acetonide and a penetration enhancer (e.g., 1% saponin, fusidate, azone, bile acid salts such as glycholate and cholate) in an ophthalmically acceptable carrier (comprising benzalkonium chloride (0.01%) as a preservative, sodium chloride, hydroxypropyl methylcellulose, dibasic sodium phosphate, polysorbate 80, citric acid and/or sodium hydroxide (to adjust pH) and purified water) to provide an ophthalmic composition of the present invention.

An appropriate amount (a volume including between about 4 and 20 mg Triamcinolone acetonide, generally between 1 microliter and 100 microliter) of the ophthalmic composition of the present invention is applied as a mist using a device of the present invention to a subject suffering from ocular inflammation. Marked reduction of inflammation is observed.

### Prophetic Example 22

### Administration of Brimonidine Tartrate

To a commercially available pharmaceutical composition including 0.15% brimonidine tartrate in an ophthalmically acceptable carrier (comprising benzalkonium chloride (0.05 mg/ml) as a preservative, citric acid, polyvinyl alcohol, sodium chloride, sodium citrate and purified water with hydrochloric acid and/or sodium hydroxide to adjust pH) such as Alphagan® P is added 0.5% escin to provide an ophthalmic composition of the present invention.

1 microliter of the ophthalmic composition of the present invention is applied three times daily as a mist using a device of the present invention to a subject suffering from ocular hypertension. Marked reduction of the intraocular pressure is observed.

### Prophetic Example 23

### Administration of Nepafenac

A composition including 0.1% nepafenac solution in a standard phosphate buffered saline (PBS) ophthalmic vehicle having a pH of 7.4 is prepared. The composition is diluted, once 1:1 with PBS to produce a 0.05% nepafenac composition (composition I) and once 1:1 with a 2% saponin composition to produce a 0.05% nepafenac/1% saponin composition (composition II).

Mice with oxygen-induced ischemic retinopathy are divided into four groups.

Each eye of the first group of mice is instilled with 100 microliters of composition I (nepafenac) four times daily.

Each eye of the second group of mice is instilled with 100 microliters of composition II (nepafenac/penetration enhancer) four times daily.

Each eyes of the third group of mice is exposed to 25 microliters of composition I nebulized (nepafenac) in a device of the present invention four times daily.

Each eyes of the fourth group of mice is exposed to 25 microliters of composition II nebulized (nepafenac/penetration enhancer) in a device of the present invention four times daily.

The mice of the second group are observed to suffer extensive irritation and continually scratch at their eyes, so the experiment with the second group is discontinued.

After 5 days of treatment, the mice of the first, third and fourth group are sacrificed, the eyes rapidly removed and frozen in OCT. The extent of ocular neovascularization (NV) in terms of mean cross-sectional area of intravitreal NV is evaluated in accordance with methods known in the art (see Takahashi et. Invest. Ophthalm. Vis. Sci. 2003, 44(1), 409-415). It is seen that the least NV occurred with the fourth group (nebulized composition II (nepafenac/penetration enhancer)) followed by the the third group (nebulized compostion I (nepafenac)) followed by the first group (instilled composition I (nepafenac)).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. That said, citation or identification of any reference in this application shall not be construed as an admission that such reference is available as prior art to the present invention.

1. A method of delivering a composition, comprising:
a) providing a pharmaceutical composition comprising one or more penetration enhancers and an ophthalmically acceptable carrier;
b) generating a mist of said composition; and
c) contacting said mist with a posterior surface of an eye of a subject in need thereof wherein said penetration enhancer comprises at least 0.05% by weight of said pharmaceutical composition.

2. The method claim 1, wherein said penetration enhancer(s) is(are) selected from the group consisting of:
Benzalkonium chloride, BL-9, digitonin, escin, acetone, acyl lactylates, acyl peptides, acylsarcosinates, alcohols, alkanolamine salts of fatty acids, alkyl benzene sulphonates, alkyl ether sulphates, alkyl sulphates, allantoin, ammonium glycyrrhizide, anionic surface-active agents, 1-substituted azacycloheptan-2-ones, benzyl benzoate, benzyl salicylate, bile salts Brij 35, Brij 78/35, butan-1,4-diol, butyl benzoate, butyl laurate, butyl myristate, buty stearate, cationic surface-active agents, cetylpyridium chloride, chenodeoxycholic acid, cholate, cholic acid, citric acid, cocoamidopropylbetaine, decamethonium, decamethonium bromide, decyl methyl sulfoxide, decyl oleate, deoxycholic acid, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adiptate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one , dodecyl dimethyl amine oxides, EDTA and disodium EDTA, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, fusidic acid, fusidate, fusidic acid derivatives, glycerol monolaurate, hexyl laurate, glycocholate, glycocholic acid, glycodeoxycholic acid, glycyrrhizic acid, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hex-2,5-diol, khellin, lamepons, lauryl alcohol, lecithin, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyethylene glycol monolaurate, polyglycerol esters, polyoxyethylated castor oil, polyoxyethylene, polyoxyethylene ethers of fatty acids such as polyoxyethylene 4-, 9-, 10-, and 23-lauryl ether, polyoxyethylene 10- and 20-cetyl ether, polyoxyethylene 10- and 20-stearyl ether, polyoxyethylene monolaurate, polyoxyethylene sorbitans, polyoxyethylene sorbitan monolaurate, polyoxy:polyoxyethylene stearate, polyoxypropylene 15 stearyl ether, poly(vinyl pyridinium chloride), propan-1-ol, propan-2-ol, propylene glycol, propylene glycol diperlargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sodium cholate, sodium glycocholate, glycocholate, sodium deoxycholate, sodium taurocholate, sodium glycodeoxycholate, sodium taurodeoxycholate, sorbitan monooleate, sorbitan monolaurate, sugar esters, sulphosuccinate, taurocholic acid, taurodeoxycholic acid, tetrahydrofuran, tetrahydrofurfural alcohol, transcutol, triethanolamine dodecyl benzene sulphonate, triethanolamine oleate, TWEEN 20, urazole, urea, urosdeoxycholic acid, saponin, saponins, Brij 78, Brij 98, paraben, sodium ursodeoxycholate, and esters, salts and mixtures thereof

3. The method of claim 1, said penetration enhancer(s) comprising at least 0.1% by weight of said pharmaceutical composition.

4. The method of claim 1, said penetration enhancer(s) comprising at least 0.2% by weight of said pharmaceutical composition.

5. The method of claim 1, said penetration enhancer(s) comprising at least 0.5% by weight of said pharmaceutical composition.

6. The method of claim 1, said penetration enhancer(s) comprising at least 1% by weight of said pharmaceutical composition.

7. The method of claim 1, said penetration enhancer(s) comprising at least 2% by weight of said pharmaceutical composition.

8. The method of claim 1, said composition further comprising one or more active pharmaceutical ingredients.

9. The method of claim 1, said active pharmaceutical ingredient(s) is(are) selected from the group consisting of alpha-2 adrenergic agonists, analgesics, anesthetics, antibiotics, antidepressants, antihistamines, antipsychotics, antivascular agents, antiviral agents, aptamers, artificial tears, beta-adrenergic blocking agents, carbonic anhydrase inhibitors, catalytic antioxidants, chemotherapeutics, cholinesterase inhibitors, corticosteroids, direct acting miotics, hormones, light-activated drugs, non-steroidal anti-inflammatory drugs, ocular lubricants, ophthalmic decongestant agents, ophthalmic antiseptics, ophthalmic antifungals, peptides, prostaglandin analogs, proteins, catalytic antioxidants), sedatives, steroid, stimulants, sulfonamides, vasoconstrictors and vasodilators.

10. the method of claim 8, said active pharmaceutical ingredient(s) comprising an alpha-2 adrenergic agonist selected from the group consisting of apraclonidine, brimonidine tartrate, dapiprazole and dipivefrin.

11. The method of claim 8, said active pharmaceutical ingredient(s) comprising an antivascular agent selected from the group consisting of anecortave acetate, pegaptanib and squalamine.

12. The method of claim 8, said active pharmaceutical ingredient(s) comprising a beta-adrenergic blocking agent selected from the consisting of betaxolol, carteolol, levobunolol, metipranolol and timolol maleate.

13. The method of claim 8, said active pharmaceutical ingredient(s) comprising a carbonic anhydrase inhibitor selected from the group consisting of acetazolamide, brinzolamide, dorzolamide, methazolamide, neptzane and unoprostone isopropyl.

14. The method of claim 8, said active pharmaceutical ingredient(s) comprising a prostaglandin analog selected from the group consisting of bimatoprost, latanoprost, travoprost and unoprostone.

15. The method of claim 1, said composition comprising at least one humectant.

16. The method of claim 15, said humectant(s) selected from the group consisting of ammonium lactate, guanidine, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine and acetamide monoethanolamine, urea, or a combination thereof.

17. The method of claim 1, said composition comprising at least one preservative.

18. the method of claim 17, said preservative(s) selected from the group consisting of alkanols, C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorobutanol, chlorocresol, citric acid, cocoa butter, coconut oil, diazolidinyl urea, diisopropyl adipate, dimethyl polysiloxane, DMDM hydantoin, disodium EDTA (ethylenediamine tetraacetate), EDTA salts, EDTA fatty acid conjugates, ethanol, fatty acids, fatty alcohols, hexadecyl alcohol, hydroxybenzoate esters, iodopropynyl butylcarbamate, isononyl iso-nonanoate, isothiazolinone, jojoba oil, lanolin oil, methylparaben, mineral oil, oleic acid, olive oil, parabens, polyethers, polyoxypropylene butyl ether, polyoxypropylene cetyl ether, potassium sorbate, propylene glycols, propylparaben, silicone oils, sodium propionate, sodium benzoate, sodium bisulfite, sorbic acid, sorbates, stearic fatty acid, vitamin E, vitamin E acetate and derivatives, esters, salts and mixtures thereof.

19. The method of claim 1, said composition comprising at least one solubilizer.

20. The method of claim 19, said solubilizer9s) selected from the group consisting of citric acid, ethylenediamine-tetraacetate, sodium meta-phosphate, succinic acid, urea, cyclodextrin, polyvinylpyrrolidone, diethylammonium-orth-benzoate, micelle-forming solubilizers, TWEENS, SPANS, polyoxyethylene sorbitan fatty acid ester, polyoxyethylene n-alkyl ethers, n-alkyl amine n-oxides, poloxamers, phospholipids and cylodextrines.
